(19) United States
(12) Patent Application Publication
(10) Pub. No.: US 2025/0057899 A1
(43) Pub. Date: Feb. 20, 2025

(54) GENE THERAPY FOR CILIOPATHIES

(71) Applicant: UCL BUSINESS LTD, London (GB)

(72) Inventors: Philip Beales, London (GB); Victor Hernandez, London (GB)

(21) Appl. No.: 18/937,430

(22) Filed: Nov. 5, 2024

Related U.S. Application Data
(63) Continuation of application No. 16/610,883, filed on Nov. 4, 2019, now Pat. No. 12,171,794.

(30) Foreign Application Priority Data
May 5, 2017 (GB) 1707212.5

(51) Int. Cl. A61K 35/761; A61K 48/00; C07K 14/47; C12N 15/86

(52) U.S. Cl. CPC A61K 35/761; A61K 48/0066; A61K 48/0083; C07K 14/4702; C12N 15/86; C12N 2750/14143; C12N 2800/22

(57) ABSTRACT

There is described a vector for treating a ciliopathy such as Bardet-Biedl syndrome, wherein the vector comprises a promoter operably linked to a ciliopathy gene, wherein the vector can provide transduction of the ciliopathy gene into multiple organs, wherein the promoter is a ubiquitous promoter which can provide expression of the ciliopathy gene in the transduced organs, and wherein the ciliopathy gene encodes a functional protein corresponding to the protein that is mutated in the ciliopathy. Also described is the use of the above vector in a method of treating a ciliopathy, the method comprising administering a therapeutically effective amount of the vector to a patient suffering from a ciliopathy.

### CROSS-REFERENCE TO RELATED APPLICATIONS

This application is a continuation of U.S. application Ser. No. 16/610,883 filed on Nov. 4, 2019, which claims priority to U.S. national phase filing under 35 U.S.C. § 371 of PCT/GB2018/051219 filed on May 4, 2018, which claims priority to Application No. GB 1707212.5 filed on May 5, 2017, each of which is hereby incorporated herein by reference in its entirety.

### REFERENCE TO A “SEQUENCE LISTING”

The instant application contains a Sequence Listing which has been submitted electronically in xml format and is hereby incorporated by reference in its entirety. Said xml copy was created on Nov. 1, 2024, is named 85853.00117.xml, and is 160 kilobytes in size.

### FIELD OF THE INVENTION

The present invention relates to gene therapy vectors for the treatment of ciliopathies, including Bardet-Biedl Syndrome.

### BACKGROUND TO THE INVENTION

The ciliopathies have recently emerged as a medically important category of disease which are caused by dysfunction of non-motile cilia found on most cells in the body (Waters & Beales, Pediatr Nephrol (2011) 26:1039-1056). Most ciliopathies share common phenotypes, including retinal degeneration. It has been predicted that there are over 100 diseases that probably arise from dysfunction of cilia whereupon more than 30 conditions have now been proven. All of these conditions are debilitating and often life-limiting and, as they are nearly all loss of function, most would benefit from gene therapy approaches to treatment. Ciliopathies have a collective prevalence of around 1 in 500 of the general population. All ciliopathies have disturbed cilia function, hence the overlap in organ involvement.

The autosomal recessive Bardet-Biedl Syndrome (BBS) is one of the best characterised ciliopathies and is associated with early onset blindness, severe obesity, complex endocrine dysfunction, cognitive impairment and renal failure. Patients born with the inherited Bardet-Biedl syndrome will experience a range of debilitating medical problems, some of which are life-limiting. Affected children will eventually go blind usually beginning in their first decade owing to a failure of the light-sensitive cells at the back of the eye (the retina). Within the first year of life they will gain an extraordinary amount of body weight which if unchecked will progress to life-threatening obesity, diabetes and high blood pressure. Many patients will also develop kidney failure (that may require dialysis treatment and/or kidney transplant) at some point in their lives and most will have some form of learning difficulties. Together these problems will impact adult patients' ability to live independently and most are unemployed. Even when diagnosed early, symptom-based treatments will only manage unpreventable complications such as retinal degeneration and obesity refractory to dietary measures.

BBS, like many ciliopathies, is an autosomal recessive genetic disorder. So far 21 genes have been found to be causative. Many of these gene products interact in multi-subunit complexes. For example, a number of these proteins form a complex called the BBSome. The BBSome is believed to mediate protein trafficking to the primary cilium. Another complex, the BBS/CCT chaperonin complex, facilitates the BBSome assembly and is composed of several BBS proteins and a number of CCT chaperonin proteins. As a result of the protein products of BBS genes physically interacting to perform a common function, mutation of many different genes cause the same unusual combination of phenotypic findings. The most common genes that are mutated in BBS patients are BBS1 (42%) and BBS10 (22%). More than 30 mutations in the BBS1 gene have been identified in people with Bardet-Biedl syndrome. The human BBS1 gene is located on the long (q) arm of chromosome 11 at position 13. Mutations in the BBS1 gene likely affect the normal formation and function of cilia. Defects in these cell structures disrupt important chemical signalling pathways during development and lead to abnormalities of sensory perception. The human BBS1 gene contains 17 exons and spans approximately 23 kb. Most BBS1 gene mutations are missense or stop mutations and the most common mutation replaces the amino acid methionine with the amino acid arginine at protein position 390 (Met390Arg or M390R). The M390R mutation accounts for approximately 80% of all BBS1 mutations. The human BBS10 gene sits on chromosome 12 and the BBS10 transcript contains only 2 exons encoding for a 723 amino acid protein. The mutations found in BBS10 patients are a mix of missense, non-sense and frameshift mutations. The most common change is C91fs with nearly 50% frequency.

So far, all current gene therapies for ciliopathies are targeted to treat a single damaged organ. For example, a number of groups have attempted to treat retinopathy of the eye using subretinal injection of gene therapy vectors (e.g. Seo et al., Invest Ophthalmol Vis Sci. 54 (9):6118-32 (2013)). However, using such an approach which directly targets a single organ would mean that a different vector would need to be custom designed for each individual target organ for multi-system disorders such as ciliopathies. If many vectors need to be used, the costs could be prohibitive and the regulatory processes cumbersome. The use of a single injection rather than multi-organ injection will be highly desirable for patients; it will be less invasive, reduce visits to the clinic and reduce the risks due to multiple treatments. Therefore, there is a need to use approaches which can address the deficiencies in some or all affected organs rather than individual organs.

Williams CL et al. (Mol Ther. 25 (4):904-916 (2017)) describes the reversal of peripheral olfactory impairment in Bardet-Biedl syndrome. As with the work discussed above, Williams describes the targeting of a single organ and does not contemplate treating multiple organs. Williams targets the olfactory sensory neurons (OSNs) in the nasal tissue of mice through the intranasal administration of a gene therapy vector containing a mouse BBS gene tagged with a fluorescent protein (GFP or mCherry). Crucially, in the mouse model used by Williams, BBS protein function is only missing in mature olfactory neurons (OSNs), so this mouse model can only show recovery in the OSN cells. This means the experiments described in Williams cannot provide any information on BBS gene expression in other cell types or tissues due to the mouse model used and cannot show multiple organ recovery.

WO 03/102141 describes the identification of a mutated BBS1 gene and various uses thereof.

### SUMMARY OF THE INVENTION

In a first aspect of the invention, there is provided a vector for treating a ciliopathy, wherein the vector comprises a promoter operably linked to a ciliopathy gene, wherein the vector can provide transduction of the ciliopathy gene into multiple organs, wherein the promoter is a ubiquitous promoter which can provide expression of the ciliopathy gene in the transduced organs, and wherein the ciliopathy gene encodes a functional protein corresponding to the protein that is mutated in the ciliopathy.

Ciliopathies are generally caused by a mutation to a single gene which results in dysfunction of non-motile cilia found on most cells in the body. Therefore, the introduction of a correct gene which expresses the functional protein compensates for the mutated gene and ameliorates the effects of the ciliopathy. The vector defined above provides transduction in multiple organs. Therefore, administration of the vector by one route of administration can be used to provide gene expression in multiple organs to ameliorate the pathologies associated with the ciliopathy. This means that it is not necessary to treat each affected organ or tissue individually as has been done before. This approach targeting multiple organs at once has not been used previously in ciliopathies and it was not contemplated that such an approach would work.

Ciliopathies are a group of disorders associated with genetic mutations encoding defective proteins, which result in abnormal formation or function of cilia. Therefore, a ciliopathy is defined as “a disorder associated with a genetic mutation encoding no protein or a defective protein, which results in abnormal formation or function of cilia”. As cilia are a component of almost all vertebrate cells, cilia dysfunction can manifest as a constellation of features that include characteristically, retinal degeneration, renal disease and cerebral anomalies. Additional manifestations include congenital fibrocystic diseases of the liver, diabetes, obesity and skeletal dysplasias. Ciliopathic features have been associated with mutations in over 40 genes.

The ciliopathy that can be treated using the vector described above can be any ciliopathy which can be treated by the expression of a functional protein corresponding to the protein that is mutated in the ciliopathy. Primarily, this is ciliopathies which result from mutations which cause loss of protein function. Expression of the functional protein restores the protein function which ameliorates the abnormal formation or function of cilia. Such ciliopathies are known to those skilled in the art. The ciliopathy that can be treated may be selected from Bardet-Biedl syndrome, Meckel-Gruber syndrome, Nephronophthisis, Senior-Loken syndrome, McKusick-Kaufman syndrome, Leber's congenital amaurosis and Joubert Syndrome. In some embodiments, the ciliopathy that is treated with the vector is selected from Bardet-Biedl syndrome, Nephronophthisis, Senior-Loken syndrome, McKusick-Kaufman syndrome and Leber's congenital amaurosis. In other embodiments, the ciliopathy that is treated with the vector is selected from Bardet-Biedl syndrome, Senior-Loken syndrome, McKusick-Kaufman syndrome and Leber's congenital amaurosis. In various embodiments, the ciliopathy that is treated with the vector is selected from Bardet-Biedl syndrome and McKusick-Kaufman syndrome. In particular embodiments, the ciliopathy that is treated with the vector is Bardet-Biedl syndrome.

The vector comprises a ciliopathy gene which encodes a functional protein corresponding to the protein that is mutated in the ciliopathy. Put another way, the ciliopathy gene encodes a functional protein corresponding to the mutated protein that causes the ciliopathy. The ciliopathy gene preferably encodes the human protein, e.g. the wild type human protein. The precise ciliopathy gene will depend on the ciliopathy to be treated and the gene which is mutated and causes the pathologies of the ciliopathy. So, for example, if a patient has a ciliopathy which is caused by a mutation in the BBS1 gene, the vector for treating this patient will comprise a ciliopathy gene which encodes a functional BBS1 protein.

In some embodiments, the ciliopathy gene encodes a functional protein selected from the BBS1, BBS2, BBS3/ARL6, BBS4, BBS5, BBS6/MKKS, BBS7, BBS8, BBS9, BBS10, BBS11/TRIM32, BBS12, BBS13/MKS1, BBS14/CEP290, BBS15/C2ORF86, BBS16/SDCCAG8, BBS17/LZTFL1, BBS18/BBIP1, BBS19/IFT27, BBS20/IFT74 and BBS21/C8ORF37 protein.

In other embodiments, the ciliopathy gene encodes a functional protein selected from the BBS1, BBS2, BBS3/ARL6, BBS4, BBS5, BBS6/MKKS, BBS7, BBS8, BBS9, BBS10, BBS11/TRIM32, BBS12, BBS14/CEP290, BBS15/C2ORF86, BBS16/SDCCAG8, BBS17/LZTFL1, BBS18/BBIP1, BBS19/IFT27 and BBS20/IFT74 protein.

In various embodiments, the ciliopathy gene encodes a functional protein selected from the BBS1, BBS2, BBS3/ARL6, BBS4, BBS5, BBS6/MKKS, BBS7, BBS8, BBS9, BBS10, BBS11/TRIM32, BBS12, BBS15/C2ORF86, BBS16/SDCCAG8, BBS17/LZTFL1, BBS18/BBIP1, BBS19/IFT27 and BBS20/IFT74 protein.

In several embodiments, the ciliopathy gene encodes a functional protein selected from the BBS1, BBS2, BBS3/ARL6, BBS4, BBS5, BBS6/MKKS, BBS7, BBS8, BBS9, BBS10, BBS12 and BBS18/BBIP1 protein.

In particular embodiments, the ciliopathy gene encodes a functional protein selected from the BBS1, BBS2, BBS3/ARL6, BBS4, BBS5, BBS6/MKKS, BBS7, BBS8, BBS9, BBS10 and BBS12protein.

In specific embodiments, the ciliopathy gene encodes a functional protein selected from the BBS1 and BBS10 protein.

In some embodiments, the ciliopathy gene encodes a functional BBS1 protein.

In other embodiments, the ciliopathy gene encodes a functional BBS10 protein.

In some embodiments, the ciliopathy that is to be treated is Bardet-Biedl syndrome and the ciliopathy gene encodes a functional protein selected from the BBS1, BBS2, BBS3/ARL6, BBS4, BBS5, BBS6/MKKS, BBS7, BBS8, BBS9, BBS10, BBS11/TRIM32, BBS12, BBS13/MKS1, BBS14/CEP290, BBS15/C2ORF86, BBS16/SDCCAG8, BBS17/LZTFL1, BBS18/BBIP1, BBS19/IFT27, BBS20/IFT74 and BBS21/C8ORF37 protein.

In other embodiments, the ciliopathy that is to be treated is Meckel-Gruber syndrome and the ciliopathy gene encodes a functional BBS13/MKS1 protein.

In various embodiments, the ciliopathy that is to be treated is Nephronophthisis and the ciliopathy gene encodes a functional BBS14/CEP290 protein.

In particular embodiments, the ciliopathy that is to be treated is Senior-Loken syndrome and the ciliopathy gene encodes a functional BBS14/CEP290 protein.

In some embodiments, the ciliopathy that is to be treated is McKusick-Kaufman syndrome and the ciliopathy gene encodes a functional BBS6/MKKS protein.

In other embodiments, the ciliopathy that is to be treated is Leber's congenital amaurosis and the ciliopathy gene encodes a functional BBS14/CEP290 protein.

In various embodiments, the ciliopathy that is to be treated is Joubert Syndrome and the ciliopathy gene encodes a functional BBS14/CEP290 protein.

The functional protein encoded by the ciliopathy gene preferably does not contain additional amino acids that are not found in the wild type protein. Any additional amino acids could interfere in the normal functioning of the protein. For example, it is preferred that the functional protein does not comprise a fluorescent protein such as green fluorescent protein (GFP) or mCherry, or tags such such as a FLAG-tag or a polyhistidine-tag.

In particular embodiments, the ciliopathy gene has the nucleotide sequence of SEQ ID NO. 1 or has at least 70% sequence identity thereto, and encodes a functional BBS1 protein. In some embodiments, the ciliopathy gene has the nucleotide sequence of SEQ ID NO. 1 or has at least 72% sequence identity thereto, and encodes a functional BBS1 protein. In a number of embodiments, the ciliopathy gene has the nucleotide sequence of SEQ ID NO. 1 or has at least 74% sequence identity thereto, and encodes a functional BBS1 protein. In other embodiments, the ciliopathy gene has the nucleotide sequence of SEQ ID NO. 1 or has at least 76% sequence identity thereto, and encodes a functional BBS1 protein. In various embodiments, the ciliopathy gene has the nucleotide sequence of SEQ ID NO. 1 or has at least 78% sequence identity thereto, and encodes a functional BBS1 protein. In particular embodiments, the ciliopathy gene has the nucleotide sequence of SEQ ID NO. 1 or has at least 80% sequence identity thereto, and encodes a functional BBS1 protein. In certain embodiments, the ciliopathy gene has the nucleotide sequence of SEQ ID NO. 1 or has at least 82% sequence identity thereto, and encodes a functional BBS1 protein. In various embodiments, the ciliopathy gene has the nucleotide sequence of SEQ ID NO. 1 or has at least 84% sequence identity thereto, and encodes a functional BBS1 protein. In some embodiments, the ciliopathy gene has the nucleotide sequence of SEQ ID NO. 1 or has at least 85% sequence identity thereto, and encodes a functional BBS1 protein. In certain embodiments, the ciliopathy gene has the nucleotide sequence of SEQ ID NO. 1 or has at least 86% sequence identity thereto, and encodes a functional BBS1 protein. In a number of embodiments, the ciliopathy gene has the nucleotide sequence of SEQ ID NO. 1 or has at least 88% sequence identity thereto, and encodes a functional BBS1 protein. In other embodiments, the ciliopathy gene has the nucleotide sequence of SEQ ID NO. 1 or has at least 90% sequence identity thereto, and encodes a functional BBS1 protein. In certain embodiments, the ciliopathy gene has the nucleotide sequence of SEQ ID NO. 1 or has at least 92% sequence identity thereto, and encodes a functional BBS1 protein. In a number of embodiments, the ciliopathy gene has the nucleotide sequence of SEQ ID NO. 1 or has at least 94% sequence identity thereto, and encodes a functional BBS1 protein. In various embodiments, the ciliopathy gene has the nucleotide sequence of SEQ ID NO. 1 or has at least 95% sequence identity thereto, and encodes a functional BBS1 protein. In certain embodiments, the ciliopathy gene has the nucleotide sequence of SEQ ID NO. 1 or has at least 96% sequence identity thereto, and encodes a functional BBS1 protein. In some embodiments, the ciliopathy gene has the nucleotide sequence of SEQ ID NO. 1 or has at least 97% sequence identity thereto, and encodes a functional BBS1 protein. In a number of embodiments, the ciliopathy gene has the nucleotide sequence of SEQ ID NO. 1 or has at least 98% sequence identity thereto, and encodes a functional BBS1 protein. In some embodiments, the ciliopathy gene has the nucleotide sequence of SEQ ID NO. 1 or has at least 99% sequence identity thereto, and encodes a functional BBS1 protein. In particular embodiments, the ciliopathy gene has the nucleotide sequence of SEQ ID NO. 1.

In particular embodiments, the ciliopathy gene has the nucleotide sequence of SEQ ID NO. 2 or has at least 70% sequence identity thereto, and encodes a functional BBS10 protein. In some embodiments, the ciliopathy gene has the nucleotide sequence of SEQ ID NO. 2 or has at least 72% sequence identity thereto, and encodes a functional BBS10 protein. In a number of embodiments, the ciliopathy gene has the nucleotide sequence of SEQ ID NO. 2 or has at least 74% sequence identity thereto, and encodes a functional BBS10 protein. In other embodiments, the ciliopathy gene has the nucleotide sequence of SEQ ID NO. 2 or has at least 76% sequence identity thereto, and encodes a functional BBS10 protein. In various embodiments, the ciliopathy gene has the nucleotide sequence of SEQ ID NO. 2 or has at least 78% sequence identity thereto, and encodes a functional BBS10 protein. In particular embodiments, the ciliopathy gene has the nucleotide sequence of SEQ ID NO. 2 or has at least 80% sequence identity thereto, and encodes a functional BBS10 protein. In certain embodiments, the ciliopathy gene has the nucleotide sequence of SEQ ID NO. 2 or has at least 82% sequence identity thereto, and encodes a functional BBS10 protein. In various embodiments, the ciliopathy gene has the nucleotide sequence of SEQ ID NO. 2 or has at least 84% sequence identity thereto, and encodes a functional BBS10 protein. In some embodiments, the ciliopathy gene has the nucleotide sequence of SEQ ID NO. 2 or has at least 85% sequence identity thereto, and encodes a functional BBS10 protein. In certain embodiments, the ciliopathy gene has the nucleotide sequence of SEQ ID NO. 2 or has at least 86% sequence identity thereto, and encodes a functional BBS10 protein. In a number of embodiments, the ciliopathy gene has the nucleotide sequence of SEQ ID NO. 2 or has at least 88% sequence identity thereto, and encodes a functional BBS10 protein. In other embodiments, the ciliopathy gene has the nucleotide sequence of SEQ ID NO. 2 or has at least 90% sequence identity thereto, and encodes a functional BBS10 protein. In certain embodiments, the ciliopathy gene has the nucleotide sequence of SEQ ID NO. 2 or has at least 92% sequence identity thereto, and encodes a functional BBS10 protein. In a number of embodiments, the ciliopathy gene has the nucleotide sequence of SEQ ID NO. 2 or has at least 94% sequence identity thereto, and encodes a functional BBS10 protein. In various embodiments, the ciliopathy gene has the nucleotide sequence of SEQ ID NO. 2 or has at least 95% sequence identity thereto, and encodes a functional BBS10 protein. In certain embodiments, the ciliopathy gene has the nucleotide sequence of SEQ ID NO. 2 or has at least 96% sequence identity thereto, and encodes a functional BBS10 protein. In some embodiments, the ciliopathy gene has the nucleotide sequence of SEQ ID NO. 2 or has at least 97% sequence identity thereto, and encodes a functional BBS10 protein. In a number of embodiments, the ciliopathy gene has the nucleotide sequence of SEQ ID NO. 2 or has at least 98% sequence identity thereto, and encodes a functional BBS10 protein. In some embodiments, the ciliopathy gene has the nucleotide sequence of SEQ ID NO. 2 or has at least 99% sequence identity thereto, and encodes a functional BBS10 protein. In particular embodiments, the ciliopathy gene has the nucleotide sequence of SEQ ID NO. 2.

In the embodiments above, the nucleotide sequence of the ciliopathy gene may be codon optimised to maximise expression of the protein. In codon optimisation, the amino acid sequence of the encoded protein remains the same so it will still be functional. It is simply the nucleotide sequence that is modified. SEQ ID NOs. 11 and 12 are codon optimised nucleotide sequences encoding BBS1, and SEQ ID NOs. 13 and 14 are codon optimised nucleotide sequences encoding BBS10. These sequences have been found to give an unexpectedly large increase in gene expression.

In particular embodiments, the ciliopathy gene has the nucleotide sequence of SEQ ID NO. 11 or has at least 70% sequence identity thereto, and encodes a functional BBS1 protein. In some embodiments, the ciliopathy gene has the nucleotide sequence of SEQ ID NO. 11 or has at least 72% sequence identity thereto, and encodes a functional BBS1 protein. In a number of embodiments, the ciliopathy gene has the nucleotide sequence of SEQ ID NO. 11 or has at least 74% sequence identity thereto, and encodes a functional BBS1 protein. In other embodiments, the ciliopathy gene has the nucleotide sequence of SEQ ID NO. 11 or has at least 76% sequence identity thereto, and encodes a functional BBS1 protein. In various embodiments, the ciliopathy gene has the nucleotide sequence of SEQ ID NO. 11 or has at least 78% sequence identity thereto, and encodes a functional BBS1 protein. In particular embodiments, the ciliopathy gene has the nucleotide sequence of SEQ ID NO. 11 or has at least 80% sequence identity thereto, and encodes a functional BBS1 protein. In certain embodiments, the ciliopathy gene has the nucleotide sequence of SEQ ID NO. 11 or has at least 82% sequence identity thereto, and encodes a functional BBS1 protein. In various embodiments, the ciliopathy gene has the nucleotide sequence of SEQ ID NO. 11 or has at least 84% sequence identity thereto, and encodes a functional BBS1 protein. In some embodiments, the ciliopathy gene has the nucleotide sequence of SEQ ID NO. 11 or has at least 85% sequence identity thereto, and encodes a functional BBS1 protein. In certain embodiments, the ciliopathy gene has the nucleotide sequence of SEQ ID NO. 11 or has at least 86% sequence identity thereto, and encodes a functional BBS1 protein. In a number of embodiments, the ciliopathy gene has the nucleotide sequence of SEQ ID NO. 11 or has at least 88% sequence identity thereto, and encodes a functional BBS1 protein. In other embodiments, the ciliopathy gene has the nucleotide sequence of SEQ ID NO. 11 or has at least 90% sequence identity thereto, and encodes a functional BBS1 protein. In certain embodiments, the ciliopathy gene has the nucleotide sequence of SEQ ID NO. 11 or has at least 92% sequence identity thereto, and encodes a functional BBS1 protein. In a number of embodiments, the ciliopathy gene has the nucleotide sequence of SEQ ID NO. 11 or has at least 94% sequence identity thereto, and encodes a functional BBS1 protein. In various embodiments, the ciliopathy gene has the nucleotide sequence of SEQ ID NO. 11 or has at least 95% sequence identity thereto, and encodes a functional BBS1 protein. In certain embodiments, the ciliopathy gene has the nucleotide sequence of SEQ ID NO. 11 or has at least 96% sequence identity thereto, and encodes a functional BBS1 protein. In some embodiments, the ciliopathy gene has the nucleotide sequence of SEQ ID NO. 11 or has at least 97% sequence identity thereto, and encodes a functional BBS1 protein. In a number of embodiments, the ciliopathy gene has the nucleotide sequence of SEQ ID NO. 11 or has at least 98% sequence identity thereto, and encodes a functional BBS1 protein. In some embodiments, the ciliopathy gene has the nucleotide sequence of SEQ ID NO. 11 or has at least 99% sequence identity thereto, and encodes a functional BBS1 protein. In particular embodiments, the ciliopathy gene has the nucleotide sequence of SEQ ID NO. 11.

In particular embodiments, the ciliopathy gene has the nucleotide sequence of SEQ ID NO. 12 or has at least 70% sequence identity thereto, and encodes a functional BBS1 protein. In some embodiments, the ciliopathy gene has the nucleotide sequence of SEQ ID NO. 12 or has at least 72% sequence identity thereto, and encodes a functional BBS1 protein. In a number of embodiments, the ciliopathy gene has the nucleotide sequence of SEQ ID NO. 12 or has at least 74% sequence identity thereto, and encodes a functional BBS1 protein. In other embodiments, the ciliopathy gene has the nucleotide sequence of SEQ ID NO. 12 or has at least 76% sequence identity thereto, and encodes a functional BBS1 protein. In various embodiments, the ciliopathy gene has the nucleotide sequence of SEQ ID NO. 12 or has at least 78% sequence identity thereto, and encodes a functional BBS1 protein. In particular embodiments, the ciliopathy gene has the nucleotide sequence of SEQ ID NO. 12 or has at least 80% sequence identity thereto, and encodes a functional BBS1 protein. In certain embodiments, the ciliopathy gene has the nucleotide sequence of SEQ ID NO. 12 or has at least 82% sequence identity thereto, and encodes a functional BBS1 protein. In various embodiments, the ciliopathy gene has the nucleotide sequence of SEQ ID NO. 12 or has at least 84% sequence identity thereto, and encodes a functional BBS1 protein. In some embodiments, the ciliopathy gene has the nucleotide sequence of SEQ ID NO. 12 or has at least 85% sequence identity thereto, and encodes a functional BBS1 protein. In certain embodiments, the ciliopathy gene has the nucleotide sequence of SEQ ID NO. 12 or has at least 86% sequence identity thereto, and encodes a functional BBS1 protein. In a number of embodiments, the ciliopathy gene has the nucleotide sequence of SEQ ID NO. 12 or has at least 88% sequence identity thereto, and encodes a functional BBS1 protein. In other embodiments, the ciliopathy gene has the nucleotide sequence of SEQ ID NO. 12 or has at least 90% sequence identity thereto, and encodes a functional BBS1 protein. In certain embodiments, the ciliopathy gene has the nucleotide sequence of SEQ ID NO. 12 or has at least 92% sequence identity thereto, and encodes a functional BBS1 protein. In a number of embodiments, the ciliopathy gene has the nucleotide sequence of SEQ ID NO. 12 or has at least 94% sequence identity thereto, and encodes a functional BBS1 protein. In various embodiments, the ciliopathy gene has the nucleotide sequence of SEQ ID NO. 12 or has at least 95% sequence identity thereto, and encodes a functional BBS1 protein. In certain embodiments, the ciliopathy gene has the nucleotide sequence of SEQ ID NO. 12 or has at least 96% sequence identity thereto, and encodes a functional BBS1 protein. In some embodiments, the ciliopathy gene has the nucleotide sequence of SEQ ID NO. 12 or has at least 97% sequence identity thereto, and encodes a functional BBS1 protein. In a number of embodiments, the ciliopathy gene has the nucleotide sequence of SEQ ID NO. 12 or has at least 98% sequence identity thereto, and encodes a functional BBS1 protein. In some embodiments, the ciliopathy gene has the nucleotide sequence of SEQ ID NO. 12 or has at least 99% sequence identity thereto, and encodes a functional BBS1 protein. In particular embodiments, the ciliopathy gene has the nucleotide sequence of SEQ ID NO. 12.

In particular embodiments, the ciliopathy gene has the nucleotide sequence of SEQ ID NO. 13 or has at least 70% sequence identity thereto, and encodes a functional BBS10 protein. In some embodiments, the ciliopathy gene has the nucleotide sequence of SEQ ID NO. 13 or has at least 72% sequence identity thereto, and encodes a functional BBS10 protein. In a number of embodiments, the ciliopathy gene has the nucleotide sequence of SEQ ID NO. 13 or has at least 74% sequence identity thereto, and encodes a functional BBS10 protein. In other embodiments, the ciliopathy gene has the nucleotide sequence of SEQ ID NO. 13 or has at least 76% sequence identity thereto, and encodes a functional BBS10 protein. In various embodiments, the ciliopathy gene has the nucleotide sequence of SEQ ID NO. 13 or has at least 78% sequence identity thereto, and encodes a functional BBS10 protein. In particular embodiments, the ciliopathy gene has the nucleotide sequence of SEQ ID NO. 13 or has at least 80% sequence identity thereto, and encodes a functional BBS10 protein. In certain embodiments, the ciliopathy gene has the nucleotide sequence of SEQ ID NO. 13 or has at least 82% sequence identity thereto, and encodes a functional BBS10 protein. In various embodiments, the ciliopathy gene has the nucleotide sequence of SEQ ID NO. 13 or has at least 84% sequence identity thereto, and encodes a functional BBS10 protein. In some embodiments, the ciliopathy gene has the nucleotide sequence of SEQ ID NO. 13 or has at least 85% sequence identity thereto, and encodes a functional BBS10 protein. In certain embodiments, the ciliopathy gene has the nucleotide sequence of SEQ ID NO. 13 or has at least 86% sequence identity thereto, and encodes a functional BBS10 protein. In a number of embodiments, the ciliopathy gene has the nucleotide sequence of SEQ ID NO. 13 or has at least 88% sequence identity thereto, and encodes a functional BBS10 protein. In other embodiments, the ciliopathy gene has the nucleotide sequence of SEQ ID NO. 13 or has at least 90% sequence identity thereto, and encodes a functional BBS10 protein. In certain embodiments, the ciliopathy gene has the nucleotide sequence of SEQ ID NO. 13 or has at least 92% sequence identity thereto, and encodes a functional BBS10 protein. In a number of embodiments, the ciliopathy gene has the nucleotide sequence of SEQ ID NO. 13 or has at least 94% sequence identity thereto, and encodes a functional BBS10 protein. In various embodiments, the ciliopathy gene has the nucleotide sequence of SEQ ID NO. 13 or has at least 95% sequence identity thereto, and encodes a functional BBS10 protein. In certain embodiments, the ciliopathy gene has the nucleotide sequence of SEQ ID NO. 13 or has at least 96% sequence identity thereto, and encodes a functional BBS10 protein. In some embodiments, the ciliopathy gene has the nucleotide sequence of SEQ ID NO. 13 or has at least 97% sequence identity thereto, and encodes a functional BBS10 protein. In a number of embodiments, the ciliopathy gene has the nucleotide sequence of SEQ ID NO. 13 or has at least 98% sequence identity thereto, and encodes a functional BBS10 protein. In some embodiments, the ciliopathy gene has the nucleotide sequence of SEQ ID NO. 13 or has at least 99% sequence identity thereto, and encodes a functional BBS10 protein. In particular embodiments, the ciliopathy gene has the nucleotide sequence of SEQ ID NO. 13.

In particular embodiments, the ciliopathy gene has the nucleotide sequence of SEQ ID NO. 14 or has at least 70% sequence identity thereto, and encodes a functional BBS10 protein. In some embodiments, the ciliopathy gene has the nucleotide sequence of SEQ ID NO. 14 or has at least 72% sequence identity thereto, and encodes a functional BBS10 protein. In a number of embodiments, the ciliopathy gene has the nucleotide sequence of SEQ ID NO. 14 or has at least 74% sequence identity thereto, and encodes a functional BBS10 protein. In other embodiments, the ciliopathy gene has the nucleotide sequence of SEQ ID NO. 14 or has at least 76% sequence identity thereto, and encodes a functional BBS10 protein. In various embodiments, the ciliopathy gene has the nucleotide sequence of SEQ ID NO. 14 or has at least 78% sequence identity thereto, and encodes a functional BBS10 protein. In particular embodiments, the ciliopathy gene has the nucleotide sequence of SEQ ID NO. 14 or has at least 80% sequence identity thereto, and encodes a functional BBS10 protein. In certain embodiments, the ciliopathy gene has the nucleotide sequence of SEQ ID NO. 14 or has at least 82% sequence identity thereto, and encodes a functional BBS10 protein. In various embodiments, the ciliopathy gene has the nucleotide sequence of SEQ ID NO. 14 or has at least 84% sequence identity thereto, and encodes a functional BBS10 protein. In some embodiments, the ciliopathy gene has the nucleotide sequence of SEQ ID NO. 14 or has at least 85% sequence identity thereto, and encodes a functional BBS10 protein. In certain embodiments, the ciliopathy gene has the nucleotide sequence of SEQ ID NO. 14 or has at least 86% sequence identity thereto, and encodes a functional BBS10 protein. In a number of embodiments, the ciliopathy gene has the nucleotide sequence of SEQ ID NO. 14 or has at least 88% sequence identity thereto, and encodes a functional BBS10 protein. In other embodiments, the ciliopathy gene has the nucleotide sequence of SEQ ID NO. 14 or has at least 90% sequence identity thereto, and encodes a functional BBS10 protein. In certain embodiments, the ciliopathy gene has the nucleotide sequence of SEQ ID NO. 14 or has at least 92% sequence identity thereto, and encodes a functional BBS10 protein. In a number of embodiments, the ciliopathy gene has the nucleotide sequence of SEQ ID NO. 14 or has at least 94% sequence identity thereto, and encodes a functional BBS10 protein. In various embodiments, the ciliopathy gene has the nucleotide sequence of SEQ ID NO. 14 or has at least 95% sequence identity thereto, and encodes a functional BBS10 protein. In certain embodiments, the ciliopathy gene has the nucleotide sequence of SEQ ID NO. 14 or has at least 96% sequence identity thereto, and encodes a functional BBS10 protein. In some embodiments, the ciliopathy gene has the nucleotide sequence of SEQ ID NO. 14 or has at least 97% sequence identity thereto, and encodes a functional BBS10 protein. In a number of embodiments, the ciliopathy gene has the nucleotide sequence of SEQ ID NO. 14 or has at least 98% sequence identity thereto, and encodes a functional BBS10 protein. In some embodiments, the ciliopathy gene has the nucleotide sequence of SEQ ID NO. 14 or has at least 99% sequence identity thereto, and encodes a functional BBS10 protein. In particular embodiments, the ciliopathy gene has the nucleotide sequence of SEQ ID NO. 14.

In various embodiments, the ciliopathy gene encodes a functional BBS1 protein having the protein sequence of SEQ ID NO 9 or at least 80% sequence identity thereto. In some embodiments, the functional BBS1 protein has the protein sequence of SEQ ID NO. 9 or at least 85% sequence identity thereto. In other embodiments, the functional BBS1 protein has the protein sequence of SEQ ID NO. 9 or at least 90% sequence identity thereto. In a number of embodiments, the functional BBS1 protein has the protein sequence of SEQ ID NO. 9 or at least 95% sequence identity thereto. In particular embodiments, the functional BBS1 protein has the protein sequence of SEQ ID NO. 9.

In other embodiments, the ciliopathy gene encodes a functional BBS10 protein having the protein sequence of SEQ ID NO 10 or at least 80% sequence identity thereto. In some embodiments, the functional BBS10 protein has the protein sequence of SEQ ID NO. 10 or at least 85% sequence identity thereto. In various embodiments, the functional BBS10 protein has the protein sequence of SEQ ID NO. 10 or at least 90% sequence identity thereto. In a number of embodiments, the functional BBS10 protein has the protein sequence of SEQ ID NO. 10 or at least 95% sequence identity thereto. In particular embodiments, the functional BBS10 protein has the protein sequence of SEQ ID NO. 10.

In the description above, the term “identity” is used to refer to the similarity of two sequences. For the purpose of this invention, it is defined here that in order to determine the percent identity of two sequences, the sequences are aligned for optimal comparison purposes (e.g., gaps can be introduced in the sequence of a first sequence for optimal alignment with a second amino or nucleic acid sequence). The nucleotide/amino acid residues at each position are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Generally, the two sequences are the same length. A sequence comparison is typically carried out over the entire length of the two sequences being compared.

The skilled person will be aware of the fact that several different computer programs are available to determine the identity between two sequences. For instance, a comparison of sequences and determination of percent identity between two sequences can be accomplished using a mathematical algorithm. In a preferred embodiment, the percent identity between two nucleic acid sequences is determined using the sequence alignment software Clone Manager 9 (Sci-Ed software-www.scied.com) using global DNA alignment; parameters: both strands; scoring matrix: linear (mismatch 2, OpenGap 4, ExtGap 1).

Alternatively, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. A further method to assess the percent identity between two amino acid or nucleic acid sequences can be to use the BLAST sequence comparison tool available on the National Center for Biotechnology Information (NCBI) website (www.blast.ncbi.nlm.nih.gov), for example using BLASTn for nucleotide sequences or BLASTp for amino acid sequences using the default parameters.

The ciliopathy gene encodes a ‘functional’ protein. This means that the protein, when expressed, has the same function and activity as the wild type human protein. This could easily be determined by one skilled in the art. The protein encoded by the ciliopathy gene may be the wild type human protein. The wild type human sequence of the various proteins discussed above are well known to those skilled in the art. For example, they can be found on the publically accessible databases of the National Center for Biotechnology Information. Further, the nucleotide sequences which encode these proteins (and which would be contained in the vector) could readily be found or determined by a person skilled in the art, for example, using the genetic code which correlates particular nucleotide codons with particular amino acids.

The promoter contained in the vector is a ubiquitous promoter which is operably linked to the ciliopathy gene so that the promoter directs expression of the ciliopathy gene in the transduced organs. A ubiquitous promoter is one which is strongly active in a wide range of cells and tissues and provides constitutive expression. Suitable ubiquitous promoters are well known to those skilled in the art. A ubiquitous promoter is not tissue specific. It provides expression in multiple tissues/organs. The ubiquitous promoter results in expression of the ciliopathy gene in the transduced organs so that the expressed protein ameliorates the pathologies associated with the ciliopathy.

Suitable ubiquitous promoters include short elongation factor promoter (EFS), CAG promoter, cytomegalovirus immediate-early promoter (CMV), Ubiquitin C promoter (UBC), phosphoglycerate kinase promoter (PGK) and beta-actin promoter, e.g. chicken beta-actin promoter (CBA). These promoters are well known to one skilled in the art. Examples of the sequences of these promoters are given as SEQ ID NO. 3, SEQ ID NO. 4, SEQ ID NO. 5, SEQ ID NO. 6, SEQ ID NO. 7, SEQ ID NO. 8 and SEQ ID NO. 45. Therefore, in some embodiments, the promoter has a sequence selected from SEQ ID NO. 3, SEQ ID NO. 4, SEQ ID NO. 5, SEQ ID NO. 6, SEQ ID NO. 7, SEQ ID NO. 8 and SEQ ID NO. 45.

In particular embodiments, the promoter is an EFS promoter which may have the nucleotide sequence of SEQ ID NO. 3 or SEQ ID NO. 46.

In some embodiments, the promoter is a CAG promoter which may have the nucleotide sequence of SEQ ID NO. 4 or SEQ ID NO. 47.

In various embodiments, the promoter is a CMV promoter which may have the nucleotide sequence of SEQ ID NO. 6 or SEQ ID NO. 45.

In certain embodiments, the promoter is a UBC promoter which may have the nucleotide sequence of SEQ ID NO. 5 or SEQ ID NO. 48.

In a number of embodiments, the promoter is a PGK promoter which may have the nucleotide sequence of SEQ ID NO. 7.

In several embodiments, the promoter is a beta-actin promoter which may have the nucleotide sequence of SEQ ID NO. 8 or SEQ ID NO. 49 .

In terms of particular combinations of elements, the vector may comprise an EFS promoter operably linked to a BBS1 gene, the vector being for the treatment of Bardet-Biedl syndrome. The BBS1 gene may be selected from SEQ ID NOs. 1, 11 and 12. For example, the vector may comprise the sequence of one of SEQ ID NOs. 15, 16 and 17. Alternatively, the vector may comprise an EFS promoter operably linked to a BBS10 gene, the vector being for the treatment of Bardet-Biedl syndrome. The BBS10 gene may be selected from SEQ ID NOs. 2, 13 and 14. For example, the vector may comprise the sequence of one of SEQ ID NOs. 30, 31 and 32.

The vector may comprise a UBC promoter operably linked to a BBS1 gene, the vector being for the treatment of Bardet-Biedl syndrome. The BBS1 gene may be selected from SEQ ID NOs. 1, 11 and 12. For example, the vector may comprise the sequence of one of SEQ ID NOs. 18, 19 and 20. Alternatively, the vector may comprise a UBC promoter operably linked to a BBS10 gene, the vector being for the treatment of Bardet-Biedl syndrome. The BBS10 gene may be selected from SEQ ID NOs. 2, 13 and 14. For example, the vector may comprise the sequence of one of SEQ ID NOs. 33, 34 and 35.

The vector may comprise a CMV promoter operably linked to a BBS1 gene, the vector being for the treatment of Bardet-Biedl syndrome. The BBS1 gene may be selected from SEQ ID NOs. 1, 11 and 12. For example, the vector may comprise the sequence of one of SEQ ID NOs. 21, 22 and 23. Alternatively, the vector may comprise a CMV promoter operably linked to a BBS10 gene, the vector being for the treatment of Bardet-Biedl syndrome. The BBS10 gene may be selected from SEQ ID NOs. 2, 13 and 14. For example, the vector may comprise the sequence of one of SEQ ID NOs. 36, 37 and 38.

The vector may comprise a CBA promoter operably linked to a BBS1 gene, the vector being for the treatment of Bardet-Biedl syndrome. The BBS1 gene may be selected from SEQ ID NOs. 1, 11 and 12. For example, the vector may comprise the sequence of one of SEQ ID NOs. 24, 25 and 26. Alternatively, the vector may comprise a CBA promoter operably linked to a BBS10 gene, the vector being for the treatment of Bardet-Biedl syndrome. The BBS10 gene may be selected from SEQ ID NOs. 2, 13 and 14. For example, the vector may comprise the sequence of one of SEQ ID NOs. 39, 40 and 41.

The vector may comprise a CAG promoter operably linked to a BBS1 gene, the vector being for the treatment of Bardet-Biedl syndrome. The BBS1 gene may be selected from SEQ ID NOs. 1, 11 and 12. For example, the vector may comprise the sequence of one of SEQ ID NOs. 27, 28 and 29. Alternatively, the vector may comprise a CAG promoter operably linked to a BBS10 gene, the vector being for the treatment of Bardet-Biedl syndrome. The BBS10 gene may be selected from SEQ ID NOs. 2, 13 and 14. For example, the vector may comprise the sequence of one of SEQ ID NOs. 42, 43 and 44.

The vector may comprise a PGK promoter operably linked to a BBS1 gene, the vector being for the treatment of Bardet-Biedl syndrome. The BBS1 gene may be selected from SEQ ID NOs. 1, 11 and 12. Alternatively, the vector may comprise a PGK promoter operably linked to a BBS10 gene, the vector being for the treatment of Bardet-Biedl syndrome. The BBS10 gene may be selected from SEQ ID NOs. 2, 13 and 14.

The vector described above can provide transduction of the ciliopathy gene into multiple organs. This can be any suitable vector and such vectors are well known to those skilled in the art. In particular embodiments, the vector can cross the blood brain barrier. This allows transduction to occur in the brain and nervous system including the eye, and also in the visceral organs and musculature. Therefore, a single vector can be used to provide gene expression in multiple organs to ameliorate the pathologies associated with the ciliopathy. This gene expression may be systemic as it can occur in multiple sites throughout the body. Further, administration of the vector by a limited number of routes can be used to provide systemic gene expression to ameliorate the pathologies associated with the ciliopathy throughout the body. This means that it is not necessary to treat each affected tissue individually. This approach targeting multiple organs at once has not been used previously in ciliopathies and it was not contemplated that such an approach would work.

The organs that can be transduced with the vector described above may be selected from the central nervous system, eye (e.g. retinal photoreceptors and retinal pigmented epithelium), heart, liver, muscle, pancreas, spleen, lung and kidney. Therefore, in some embodiments, the vector provides transduction of the ciliopathy gene into multiple organs selected from the central nervous system, eye, heart, liver, muscle, pancreas, spleen, lung and kidney. In other embodiments, the vector provides transduction of the ciliopathy gene into at least three organs selected from the central nervous system, eye, heart, liver, muscle, pancreas, spleen, lung and kidney. In various embodiments, the vector provides transduction of the ciliopathy gene into at least four of the stated organs. In a number of embodiments, the vector provides transduction of the ciliopathy gene into at least five of the stated organs. In some embodiments, the vector provides transduction of the ciliopathy gene into at least six of the stated organs. In other embodiments, the vector provides transduction of the ciliopathy gene into at least seven of the stated organs. In various embodiments, the vector provides transduction of the ciliopathy gene into at least eight of the stated organs. In particular embodiments, the vector provides transduction of the ciliopathy gene into the central nervous system, eye, heart, liver, muscle, pancreas, spleen, lung and kidney. In certain embodiments, the vector provides transduction of the ciliopathy gene into at least the central nervous system (e.g. the brain) and the eye. In various embodiments, the vector provides transduction of the ciliopathy gene into at least the central nervous system (e.g. the brain), the eye, and one of the liver, kidney and spleen. In some embodiments, the vector provides transduction of the ciliopathy gene into at least the central nervous system (e.g. the brain), the eye and the liver. In particular embodiments, the vector provides transduction of the ciliopathy gene into at least the central nervous system (e.g. the brain), the eye, the liver, the kidney and the spleen.

Suitable vectors include adeno-associated virus-8 (AAV8) and adeno-associated virus-9(AAV9) and also other AAVs (e.g. AAV2) which have been pseudotyped with the capsid proteins from AAV8 or AAV9. Such vectors are described in WO 2005/033321. Other suitable vectors include AAV-PHP.A and AAVPHP.B (Nature Biotechnology 34, 204-209 (2016)), AAV9.47 (Hum Gene Ther. 2016 July;27(7): 497-508), AAV-B1 (Mol. Ther. 24, 1247-1257), AAV8 (Y733F) (Mol Ther 2009; 17:463-471) and AAV2-TT (described in WO2015/121501). Lentiviral vectors can also be used, for example, as described in Trends in Molecular Medicine, April 2016, Vol. 22, No. 4 and Ther Deliv. 2010 October; 1(4): 517-534.

In some embodiments, the vector is an AAV vector such as AAV8, AAV9, AAV vectors pseudotyped with the capsid proteins from AAV8 or AAV9, AAV-PHP.A, AAV-PHP.B, AAV9.47, AAV-B1, AAV8 (Y733F) or AAV2-TT. In other embodiments, the vector is selected from AAV8, AAV9, AAV vectors pseudotyped with the capsid proteins from AAV8 or AAV9, AAV-PHP.A, AAV-PHP.B, AAV9.47 and AAV-B1. In various embodiments, the vector is selected from AAV8, AAV9, AAV vectors pseudotyped with the capsid proteins from AAV8 or AAV9, AAV-PHP.A and AAV-PHP.B. In a number of embodiments, the vector is selected from AAV8, AAV9, AAV vectors pseudotyped with the capsid proteins from AAV8 or AAV9, and AAV-PHP.B. In particular embodiments, the vector is selected from AAV8, AAV9, and AAV vectors pseudotyped with the capsid proteins from AAV8 or AAV9. In some embodiments, the vector is selected from AAV8 and AAV vectors pseudotyped with the capsid proteins from AAV8 (e.g. AAV2 pseudotyped with the capsid proteins from AAV8 (AAV2/8)). In other embodiments, the vector is selected from AAV9 and AAV vectors pseudotyped with the capsid proteins from AAV9 (e.g. AAV2 pseudotyped with the capsid proteins from AAV9 (AAV2/9)).

The adeno-associated viral vector may be a recombinant adeno-associated viral (rAAV) vector. AAV is a member of the family Parvoviridae which is described in Kenneth I. Berns, “Parvoviridae: The Viruses and Their Replication,” Chapter 69 in Fields Virology (3d Ed. 1996).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and-3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

In an AAV suitable for use as a gene therapy vector, the vector genome typically comprises a nucleic acid (e.g. a ciliopathy gene) to be packaged for delivery to a target cell. According to this particular embodiment, the heterologous nucleotide sequence is located between the viral ITRs at either end of the vector genome. In further preferred embodiments, the parvovirus (e.g. AAV) cap genes and parvovirus (e.g. AAV) rep genes are deleted from the template genome (and thus from the virion DNA produced therefrom). This configuration maximizes the size of the nucleic acid sequence(s) that can be carried by the parvovirus capsid.

According to this particular embodiment, the nucleic acid is located between the viral ITRs at either end of the substrate. It is possible for a parvoviral genome to function with only one ITR. Thus, in a gene therapy vector based on a parvovirus, the vector genome is flanked by at least one ITR, but, more typically, by two AAV ITRs (generally with one either side of the vector genome, i.e. one at the 5′ end and one at the 3′ end). There may be intervening sequences between the nucleic acid in the vector genome and one or more of the ITRs.

Generally, the ciliopathy gene (i.e. the nucleotide sequence encoding a functional protein corresponding to the protein that is mutated in the ciliopathy (for expression in the mammalian cell)) will be incorporated into a parvoviral genome located between two regular ITRs or located on either side of an ITR engineered with two D regions.

In one aspect, the invention provides a pharmaceutical composition comprising a vector as described above and one or more pharmaceutically acceptable excipients. The one or more excipients include carriers, diluents and/or other medicinal agents, pharmaceutical agents or adjuvants, etc.

The invention also provides a method of treating a ciliopathy comprising administering a therapeutically effective amount of a vector as described above to a patient suffering from a ciliopathy. Preferably, the patient is human.

When the ciliopathy is “treated” in the above method, this means that one or more symptoms of the ciliopathy are ameliorated. It does not mean that the symptoms are completely remedied so that they are no longer present in the patient, although in some methods, this may be the case. The method of treating results in one or more of the symptoms of the ciliopathy being less severe than before treatment. The method of treating may result in a plurality of the symptoms of the ciliopathy being less severe than before treatment. The amelioration of the symptoms occurs in multiple organs due to transduction and gene expression in multiple organs.

A “therapeutically effective amount” refers to an amount effective, at dosages and for periods of time necessary, to achieve the desired therapeutic result, such as raising the level of functional protein in a subject (so as to lead to a level sufficient to ameliorate the symptoms of the ciliopathy).

The method of treatment causes an increase in the level of functional protein in the subject. In some embodiments, the method of treatment causes an increase in the level of functional protein to about a normal level (i.e. the level found in a normal healthy subject). In one embodiment, the method of treatment causes an increase in the level of functional protein to, at most, normal levels.

The vector may be administered in any suitable way so as to allow expression of the ciliopathy gene in multiple organs. In particular embodiments, a single administration of the vector can be used to provide gene expression to ameliorate the pathologies associated with the ciliopathy. Administration of the vector may provide systemic gene expression to ameliorate the pathologies associated with the ciliopathy throughout the body. The vector may be administered intravenously or intracranially. In particular embodiments, the vector is administered intravenously. In some embodiments, the vector is administered intracranially. In various embodiments, the vector is administered intravenously and intracranially.

The vector may be administered intrathecally. This can be alone or in addition to intravenous and/or intracranial administration.

Intracranial administration is the direct delivery of the vector to specific areas of the brain by means of a stereotaxic injection. Intracranial administration does not include subretinal administration, e.g. subretinal injection.

Further, the vector should preferably not be administered intranasally. The nasal route of administration can restrict expression of the vector to a small subset of nasal cells and does not allow the vector to target the main affected tissues in other parts of the body. In addition, the nasal route does not allow long term sustained expression of the transgene due to rapid replacement of the small subset of nasal cells.

If the vector is administered by multiple routes of administration, for example, intravenously and intracranially, the vector is administered at both sites on the same day. In some embodiments, the multiple administrations are given within the space of six hours, within the space of four hours, or even within the space of two hours. In some embodiments, the multiple administrations are given simultaneously.

The vector may be administered at a single point in time. For example, a single injection may be given. If the vector is administered by multiple routes of administration, for example, intravenously and intracranially, the vector is administered at both sites only once (and at least on the same day as described above). No further administrations are given subsequently.

Further, the invention provides the vector described above for use in therapy, for example, in the treatment of a ciliopathy.

In addition, the invention provides the use of the vector as described above in the manufacture of a medicament for treating a ciliopathy.

All patent and literature references cited in the present specification are hereby incorporated by reference in their entirety.

### BRIEF DESCRIPTION OF THE DRAWINGS

The invention will now be described in detail by way of example only with reference to the figures which are as follows:

FIG. 1: Restriction digestion. Plasmid DNA of AAV2/8, Helper Virus and BBS1 Digested Using Xhol. Gel electrophoresis showing plasmid DNA of each helper plasmid (pHGTI, lane 2), AAV2/8 plasmid (pLT-AAV2/8, lane 3) and BBS1 plasmid (pAV-EFS-BBS1, lane 4) digested with restriction enzyme Xhol on 1% agarose gel. HyperLadder is seen in lane 1. Plasmids were used to create hAAV2/8-EFS-BBS1. The band sizes for the helper plasmid were 6318 bp and 11549 bp (lane 2) and for the AAV2/8 plasmid, 186 bp, 2109 bp and 4844 bp (lane 3), respectively. For pAV-EFS-BBS1, the band sizes were 236 bp, 643 bp, 1195 bp and 5078 bp (lane 4).

FIG. 2: human BBS1 Transfections of 293T Cells. Protein and mRNA expression following transfection of 293T with EFS-BBS1 plasmid. A) 2% agarose gel following reverse-transcription PCR from mRNA extracted from transfected and untransfected 293T cells. Lane 1 marker and lanes 2, 4 and 6 show transfected cDNA; lanes 3, 5 and 7, transfected cells without reverse transcription; lanes 8, 10 and 12, non-transfected cDNA; and lanes 9, 11 and 13, untransfected, no-reverse transcriptase controls. Lane 14 shows a ddH20 sample for PCR as the/a negative control. B) BBS1 protein expression following transfection was visible in 293T cells following transfection. Lanes 2, 3 and 4 are transfected cells, and lanes 5, 6 and 7 are untransfected. Protein expression of HSP90B and GAPDH was used as internal control.

FIG. 3: Expression of human BBS1 in the retina and CNS (Central Nervous System). RT-PCR showing transgene human BBS1 expression. P35 animals after intracranial and systemic delivery. Intracranial injection of AAV2/8-EFS-BBS1 (I.C.) shows clear expression of human BBS1 alongside mouse Bbs1 in the brain and eye (black arrows), indicating good transduction and expression. A clear expression of human BBS1 is observed in the eyes of intravenously (systemically) injected animals (I.V.) (white arrows). Specific primers to differentiate mouse and human BBS1 were designed. mBbs1 (mouse Bbs1); AAV-hBBS1 (transduced human BBSI); β-actin (β-actin positive control); Nephrin (negative control).

FIG. 4. Expression of human BBS1 by RT-PCR in the retina and CNS (Central Nervous System) at P180 (180 days post-injection) after P0 intracranial and systemic delivery in mice. A) A clear expression of human BBS1 is observed in the eyes and brain of intravenously (systemically) injected animals (I.V.). B) Intracranial injection of AAV2/8-EFS-BBS1 (I.C.) shows clear expression of human BBS1 in the brain, indicating good transduction and expression. Specific human primers to differentiate mouse and human BBS1 were designed. M390R=Bbs1^{M390R/M390R} animals. +/+=Wild type animals. RT-=RT-PCR negative control.

FIG. 5. Phenotypical expression in treated and untreated Bbs1^{M390R/M390R} demonstrates reduction in weight in P0 intracranial treated animals. When treated intracranially with the AAV2/8 EFS-BBS1 construct, Bbs1^{M390R/M390R} animals show statistically significant recovery of weight back to wild-type levels (see p-values) in males and females. IC=Intracranial. Het=Heterozygous animals, Bbs1^{M390R/M390R}. WT=Wild-type animals. M390R=Homozygous animal Bbs1^{M390R/M390R}. Error Bars show Standard error of the Mean (S.E.M). p-values were obtained after non-linear curve fitting, (Gompertz growth curve fitting), followed by ANOVA and Tukey's test.

FIG. 6. Phenotypical expression in treated and untreated Bbs1^{M390R/M390R} demonstrates reduction in weight in P0 systemically treated animals. When treated intravenously with AAV2/8 EFS-BBS1 construct, Bbs1^{M390R/M390R} animals show an attenuated weight gain to wild-type levels (see p-values) in males and females. IV=Intravenous. Het=Heterozygous animals, Bbs1^{M390R/M390R}. WT=Wild-type animals. M390R=Homozygous animal Bbs1^{M390R/M390R}. Error Bars show Standard error of the Mean (S.E.M). p-values were obtained after non-linear curve fitting, (Gompertz growth curve fitting), followed by ANOVA and Tukey's test.

FIG. 7. Graphical representation of the Gompertz growth nonlinear regression analysis. Gompertz growth fitted the best of all tested nonlinear regression curves function. A. Intracranially P0 treated males. B. Systemically P0 treated males. C. Intracranially P0 treated females. D. Systemically P0 treated females. I.C.=Intracranial. I.V.=Intravenous.

FIG. 8. Serum leptin plasma concentration/weight. AAV2/8-EFS-BBS1 IV and IC Bbs1^{M390R/M390R} treated animals have reduced serum leptin concentrations 6 months after treatment. M390R=Homozygous animal Bbs1^{M390R/M390R}. WT=Wild-type animals. IC=Intracranially treated. IV=Intravenously treated.

FIG. 9. Eosin-Haematoxylin retinal sections showing AAV2/8-EFS-BBS1 IV treated Bbs1^{M390R/M390R} animals have attenuated loss of photoreceptors 6 months after treatment. For quantification view FIG. 11. ONL=Outer nuclear layer. IN=Inner nuclear layer. All images were taken with a x40 objective.

FIG. 10. Eosin-Haematoxylin retinal sections showing AAV2/8-EFS-BBS1 IC treated Bbs1^{M390R/M390R} animals have attenuated loss of photoreceptors 6 months after treatment. For quantification view FIG. 11. ONL=Outer nuclear layer. IN=Inner nuclear layer. All images were taken with a x40 objective.

FIG. 11. Quantification of the outer nuclear layer section retinal thickness. The number of ONL nuclei recovered in Bbs1^{M390R/M390R} treated animals is double compared to untreated animals.

FIG. 12. A map of the vector contain EFS and human BBS1 (EFS-hBBS1) used in the following examples with the main features indicated. This vector was used to produce the AAV2/8-EFS-BBS1 virus. In certain embodiments of the invention, the promoter, ciliopathy gene and restriction sites can be different depending on the final product.

FIG. 13. Expression of human BBS1 by RT-PCR in the eye and brain at 52 weeks after P0 intracranial AAV2/8-EFS-BBS1 delivery. A clear expression of human BBS1 is observed in the eyes and brain of injected animals after intracranial injection of AAV2/8-EFS-BBS1 (IC treated), indicating good transduction and long lasting expression. Specific human primers to differentiate mouse and human BBS1 were designed, and no detection of mouse Bbs1 was observed. M390R=Bbs1^{M390R/M390R}, WT, wild type animals, UT, untreated; IC treated, intracranially treated.

FIG. 14. Construct efficacy after a year of injection. 12 month after perinatal intracranial treatment with the construct AAV2/8 EFS-BBS1, Bbs1^{M390R/M390R} treated males continue to demonstrate a reduction in weight compared with wild-type untreated littermates. WT=Wild-type animals. M390R=Homozygous animal Bbs1^{M390R/M390R}. Error Bars show Standard error of the Mean (S.E.M).

FIG. 15. Levels of circulating leptin 52 weeks post-treatment. Male and female Bbs1^{M390R/M390R} animals show elevated levels of leptin. Male AAV2/8-EFS-BBS1 treated animals have the same leptin levels as untreated wild-type animals. Treated Bbs1^{M390R/M390R} vs untreated wild-types are not significantly different (p-value 0.647). Untreated Bbs1^{M390R/M390R} vs treated Bbs1^{M390R/M390R} have a significant p-value of 0.027. Female AAV2/8-EFS-BBS1 treated animals show a significant reduction of their leptin levels compared with untreated Bbs1^{M390R/M390R} animals (p-value 0.041). M, male; F, females; WT, wild type; HOM, Bbs1^{M390R/M390R}, UT, Untreated; IC, intracranial.

FIG. 16. Real Time PCR showing expression of BBS1 after individual transfections with all constructs from SEQ ID NO. 15 to SEQ ID NO. 29. After transfection of the constructs, total RNA was extracted and levels of RNA were quantified and normalised for the construct with SEQ ID NO. 15 (EFS-WTBBS1).

FIG. 17. Real Time PCR showing expression of BBS10 after individual transfections with all constructs from SEQ ID NO. 30 to SEQ ID NO. 44. After transfection of the constructs, total RNA was extracted and levels of RNA were quantified and normalised for the construct with untransfected HEK293T total mRNA.

FIG. 18. Western Blots showing expression of BBS1 protein after individual transfections with constructs with SEQ ID NO. 15 to SEQ ID NO. 29. Blots were quantified using Image J and all BBS1 expression levels were normalised for SEQ ID NO. 15 (EFS-WTBBS1).

FIG. 19. Blots from FIG. 18 were quantified using Image J and all BBS1 expression levels were normalised for SEQ ID NO. 15 (EFS WT BBS1). Nearly all constructs and new BBS1 sequences showed an increased BBS1 protein expression compared with the initial EFS-WTBBS1 construct.

FIG. 20. RT-PCR for COSEQ1-BBS1 from samples of P0 AAV2/9-CAG-COSEQ1-BBS1 intracranially injected animals. Bbs1^{+/M390R} animals were culled at 8, 14 and 40 days post injection with P0 AAV2/9-CAG-COSEQ1-BBS1. Total RNA was extracted and cDNA synthesised. A PCR for the COSEQ1-BBS1 with an expected band of 188 nucleotides was performed and expression of the gene expression was detected in the brain and in the eye. P, days postinjection, C-control vehicle injected, bp. Base pairs.

### DETAILED DESCRIPTION OF THE INVENTION

The inventors have developed a single vector which is administered by a simple injection and which targets multiple organs at once. This approach is simpler and certainly more cost-effective than an alternative multi-vector approach. Restoring function in more than one organ (e.g. vision and weight lowering) would be life-changing by improving the health and quality-of-life of ciliopathy patients.

Given the broad range of organ involvement in ciliopathies, a multi-organ therapy is required that would address both the central nervous system and visceral symptoms. Furthermore, the early appearance of symptoms in patients in infancy would ideally require such therapy to be administered as early as possible. The delivery of a single-therapy during the neonatal period will target multiple organs effectively, prevent irreversible pathology and be cost-effective.

To achieve these aims, the inventors have used a gene therapy based protocol that utilises the adeno-associated virus to achieve multi-organ therapy. Until recently, multi-organ gene delivery had been difficult due to the inability to find viral vectors that can target peripheral organs and cross the blood-brain barrier. However, the discovery that AAV8 and AAV9 can cross the blood-brain barrier and mediate highly efficient gene delivery to the central nervous system of mice (Foust KD et al. Nat Biotechnol. 2009;27(1):59-65) and non-human primates (Foust KD et al. Nat Biotechnol. 2010; 28(3): 271-4; Bevan AK et al. Mol Ther. 2011; 19(11):1971-80) has changed the field's perspective. It is now feasible to consider simplifying the administration of vectors so that multiple organs can be treated with fewer routes of administration rather than using a one administration per organ regime. For example, it has been demonstrated that intravenous administration of AAV8 or AAV9 carrying the green fluorescent protein (GFP) gene to newborn mice leads to extensive and global transduction of the brain and nervous system including the eye. Furthermore, the inventors have data showing that this approach also leads to extensive systemic transduction including the visceral organs and musculature (FASEB J. 2015 September;29 (9): 3876-88.).

A multisystem and progressive disorder that presents symptoms in infancy such as Bardet-Biedl Syndrome is an ideal candidate for neonatal therapeutic AAV delivery. As the most common mutations causing BBS are found in BBS1, the inventors have tested an AAV8 and AAV9 vector carrying the human gene BBS1 in one BBS murine model of disease, Bbs1^{M390R/M390R}. This model is a “knock-in” of the common mutation and is well validated and characterised to recapitulate the human BBS phenotype (including blindness and obesity). Transcription of the exogenous BBS1 gene is driven by the mammalian ubiquitous EFS promoter, widely expressed in targeted organs. This approach exploits the capability of AAV8 and AAV9 to cross the blood brain barrier, where neuronal defects such as the retina, the hippocampus/dentate gyrus or the hypothalamic appetite centre can then be targeted, hence restoring retinal function and obesity respectively. Successful systemic gene transfer in these disease models provides the necessary proof-of-principle, optimal dosage information, efficacy of restitution, and toxicity and safety profiles of the vectors in advance of clinical trials for patients.

The inventors cloned the human BBS1 cDNA under the control of the short elongation factor promoter (EFS) into an AAV-2 vector pseudotyped with the capsid proteins from adeno-associated virus-8 (AAV2/8). These data demonstrate the EFS-BBS1 construct efficiently transfects and expresses human BBS1 in HEK293T cells. After viral production and infection via intracranial delivery or systemic (IV) delivery of P0 pups, good transduction was shown in the retina and brain. No toxicological effect in treated mice were observed. The inventors were able to demonstrate the obesity and retinal phenotype can be rescued to a high degree when mutant Bbs1^{M390R/M390R} animals were treated.

The BBS1 nucleotide and amino acid sequence is highly conserved between human and mouse (92.2%). A knock-in mouse was produced carrying the M390R mutation in the Bbs1 gene-the most common mutation in patients (Proc Natl Acad Sci USA. 2007 Dec. 4; 104(49): 19422-19427). Sequential histology of the Bbs1^{M390R/M390R} mice retinae shows progressive degeneration, of inner and outer segments (IS and OS), that is slow and complete by 6 months after birth. Electroretinograms (ERG) of Bbs1^{M390R/M390R} knock-in mice show significant attenuation in the a-and b-waves and a lower attenuation of the c-waves, suggesting the degeneration predominantly affects cone and rod photoreceptor cells and not the retinal pigmented epithelium (RPE). In addition, Bbs1^{M390R/M390R} mice also develop obesity associated with high serum levels of adipocyte-derived leptin hormone suggesting leptin resistance, increased food intake and decreased locomotor activity. Also, numerous neuroanatomical defects are detected including a reduction in the size of the corpus striatum and hippocampus, areas important in cognition and learning. These phenotypes recapitulate the human disease making the mouse model ideal for assessing novel treatments. Both mouse Bbs10 and human BBS10 genes are encoded by two exons. Their proteins are conserved with 67% identical amino acid sequences. The Bbs10 null (Bbs10^{-/-}) mouse is lacking completely exon 2 of Bbs10. Bbs10^{-/-} mice display typical BBS phenotype with a perinatal period with a runting and with an obesity onset from 8onwards and are overweight at the third month of life. Bbs10^{-/-} mice also develop hyperphagia and high levels of circulating leptin. Bbs10^{-/-} mice develop severe retinal degeneration, with a clear loss of the inner IS and OS of the photoreceptors and the ONL by 3 months of age ((ilia 2015 4:10).

### Materials and Methods

A construct has been produced where human BBS1 cDNA (SEQ ID NO. 1-NM_024649.4) has been cloned under the control of the EFS promoter (Human eukaryotic translation elongation factor 1 α1 short promoter) in an AAV2/8 viral plasmid. As the aim of the project was to move towards viral gene therapy for Bardet-Biedl syndrome 1 (BBS1), an adeno-associated virus (AAV) containing the human wild-type BBS1 cDNA and driven by an elongation factor-1α short (EFS) promoter was produced. For virus production, usual methods were used. 4000 cm² of HEK293T cell monolayer cells were transfected with the EFS-BBS1-AAV-ITR containing plasmid, AAV2 Rep-Cap plasmid and the helper plasmid. Once showing cytopathic effects, cells were harvested and lysed to release the virus. The adeno-associated virus was purified by centrifugation using two sequential caesium chloride gradients. The final product was desalted, titered both spectrophotometrically for viral particles and plaque formation assay for PFU/IFU.

The inventors also cloned BBS10 wild-type cDNA (SEQ ID NO. 2) under the control of the EFS promoter, and also completely novel codon optimised sequences for BBS1 (SEQ ID NOs. 11 and 12) and BBS10 (SEQ ID NOs. 13 and 14) to improve levels of gene expression and efficacy. Novel sequences were cloned under the control of EFS, CAG, CMV, CBA, UBC promoters. All possible combinations of the described promoters and described BBS1 and BBS10 sequences were cloned into pAV-AAV-ITR containing plasmids. Promoters were cloned between Spel and EcoRI restriction sites, followed by inserting the BBS coding sequences, 3′downstream from the promoters, with EcoRI and SalI restriction enzymes. Clones were sequenced to check unwanted mutations in promoter and coding regions. All sequences containing the promoter and gene sequence are set out as SEQ ID NO. 15 to SEQ ID NO. 44.

To test improved gene expression, HEK293T cells were transfected with all different constructs; pAV-EFS-WTBBS1, pAV-EFS-COSEQ1-BBS1, pAV-EFS-COSEQ2-BBS1, pAV-UBC-WTBBS1, pAV-UBC-COSEQ1-BBS1, pAV-UBC-COSEQ2-BBS1, pAV-CMV-WTBBS1, pAV-CMV-COSEQ1-BBS1, pAV-CMV-COSEQ2-BBS1, pAV-CBA-WTBBS1, pAV-CBA-COSEQ1-BBS1, pAV-CBA-COSEQ2-BBS1, pAV-CAG-WTBBS1, pAV-CAG-COSEQ1-BBS1, pAV-CAG-COSEQ2-BBS1, pAV-EFS-WTBBS10, pAV-EFS-COSEQ1-BBS10, pAV-EFS-COSEQ2-BBS10, pAV-UBC-WTBBS10, pAV-UBC-COSEQ1-BBS10, pAV-UBC-COSEQ2-BBS10, pAV-CMV-WTBBS10, pAV-CMV-COSEQ1-BBS10, pAV-CMV-COSEQ2-BBS10, pAV-CBA-WTBBS10, pAV-CBA-COSEQ1-BBS10, pAV-CBA-COSEQ2-BBS10, pAV-CAG-WTBBS10, pAV-CAG-COSEQ1-BBS10, pAV-CAG-COSEQ2-BBS10, using 1 g/1 of DNA, using a Lipofectamine 2000 protocol.

Cells were harvested for total mRNA with 0.5 ml of Trizol. Total mRNA was quantified and Real Time PCR was performed using 1 g of mRNA for each transfection. Specific primers for each sequence were used for each construct to quantify levels of human BBS1 expression. Ct values Expression levels were normalised for EFS-BBS1 for all BBS constructs and for untransfected samples for BBS10 constructs.

In a separate experiment, cells were also transfected for BBS1 protein expression. RIPA Buffer was used to extract total protein and total protein quantified for each transfection. 1 g/1 of sample protein for each transfection was loaded in a 4-20% acrylamide gel. A western blot was performed with a specific antibody against BBS1 and the gel was scanned and analysed. As a loading control a second western blot was performed with a GAPDH antibody. Blots were quantified by normalising first for GAPDH, for each lane, and then to EFS-WTBBS1 expression, for each gel.

### Virus Administration and Titer

Timed matings were prepared between Bbs1^{M390R/+} males and Bbs1^{M390R/+} females. P0 pups were genotyped for sex and Bbs/genotype. The adenoviral-associated vector was given via two routes of administration in P0 animals a) intracranially (5 μl of 3.5×10¹³ vg/ml (vector genomes/ml)) and systemically (IV) (20 μl of 3.5×10¹³ vg/ml). Systemic injections were executed through the temporal face vein.

The inventors injected 3 different groups of animals; Bbs1^{M390R/M390R} animals, wild-type and heterozygous. Uninjected controls have been used as a control for each group. A total of n=6animals/group were used. Treated animals do not show any physical or behaviour distress after 6 months post-injection.

Based on the results with the codon optimised sequences and constructs, the inventors decided to test the capacity of the new constructs to deliver and express human BBS1 to different tissues. The authors tested a new virus capsid (AAV2/9), a new CAG promoter and the new CAG-COSEQ1-BBS1 construct (SEQ ID NO. 28). AAV2/9 vectors containing the CAG-COSEQ1-BBS1 construct (SEQ ID NO. 28) were produced and tested for effectiveness by dosing P0 neonatal pups to restore Bbs1 activity. Bbs1^{M390R/M390R} neonates were injected intracranially with 0.175×10¹² vg per animal, in a 5 1 injection. Control, Bbs1^{+/M390R} and WT animals were also injected with the AAV2/9-CAG-COSEQ1-BBS1 or vehicle and tested for human BBS1 expression at 14 and 40 days after injections.

### Results

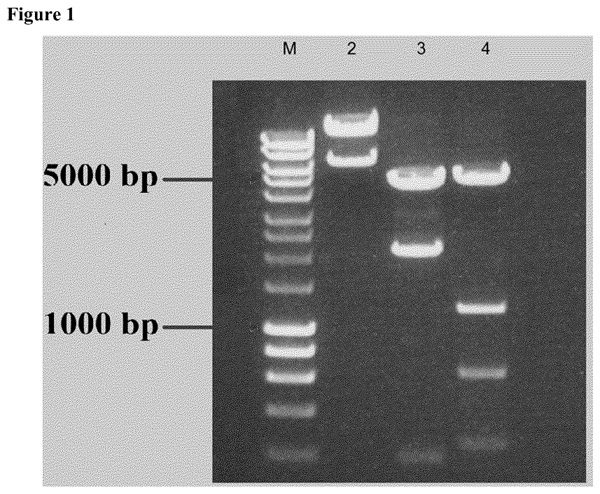

The inventors show for the first time the treatment of multiple tissues affected by a ciliopathy disorder, Bardet-Biedl Syndrome, using gene therapy techniques. The human BBS1 cDNA under the control of the ubiquitous promoter EFS transduced expression of BBS1 protein in an AAV2/8 vector. FIG. 1 show the right size after digestion of the cloned pAV-EFS-BBS1 (lane 4), demonstrating a correct cloning. In order to create this AAV, the producer plasmids pHGTI, pLT-AAV2-8, and pAV-EFS-BBS1 were obtained. The helper plasmid was pHGTI, which contains sequences coding for herpes simplex virus proteins. These are necessary for the efficient production of AAV. The pLT-AAV2-8 plasmid contains sequences coding for AAV rep and cap genes from AAV2 and AAV8, respectively. The rep gene is necessary for AAV replication, and the cap genes code for capsid proteins, which determine the tropism of the AAV. Finally, the pLT-AAV2-8 plasmid contains AAV inverted terminal repeat sequences, along with the BBS1 cDNA, which is controlled by the EFS promoter. It is these sequences that are packaged into the AAV and delivered to the cells.

To assess if the producer plasmids obtained were as expected, a restriction enzyme digest was carried out by digesting pLT-AAV2-8, pHGTI, and pAV-EFS-BBS1 plasmid DNA (FIG. 1). The band sizes of pLT-AAV2-8 plasmid were 186 bp, 2109 bp and 4844 bp, respectively (lane 3, FIG. 1). For the transgene pAV-EFS-BBS1 construct, the band sizes were 236 bp, 643 bp, 1195 bp and 5078 bp (lane 4, FIG. 1). For the helper plasmid, the band sizes visible were 6318 bp and 11549, respectively, (lane 2, FIG. 1) which verified that there was no additional, unwanted DNA present in the plasmids.

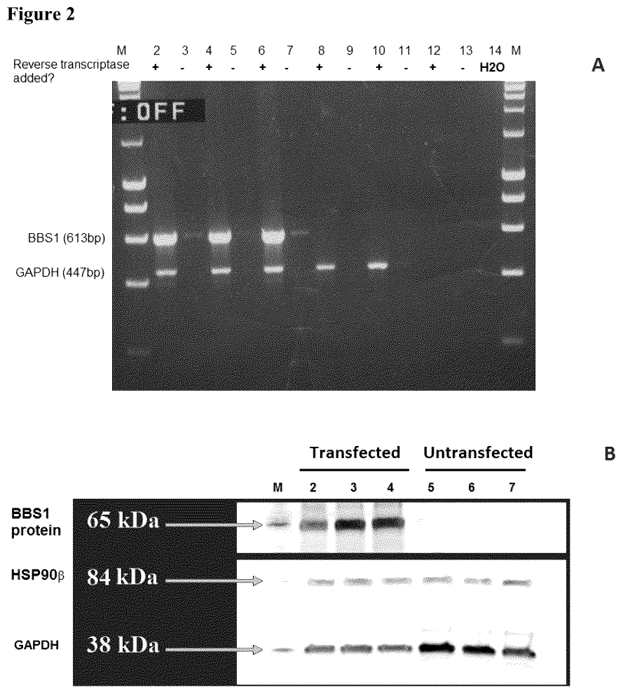

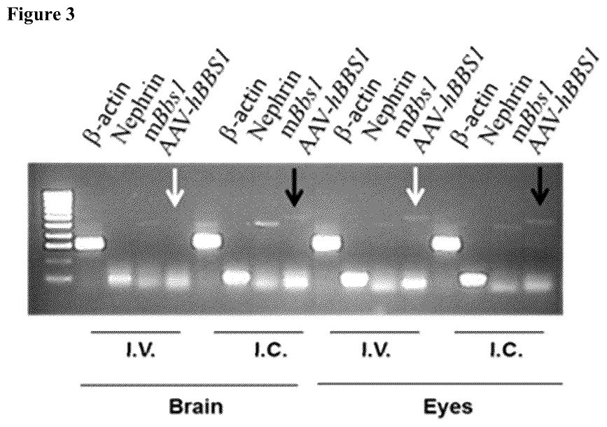

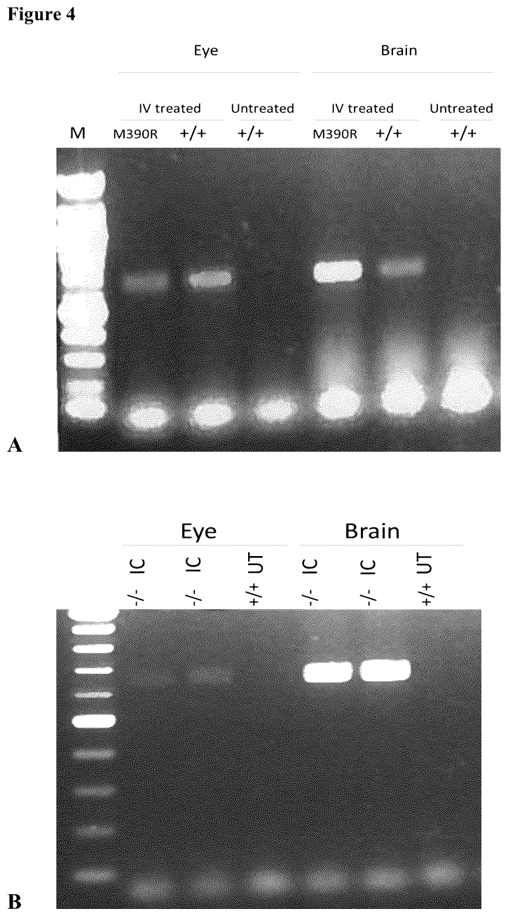

High expression of BBS1 was observed when HEK293T cells were transfected with pAV-EFS-BBS1 plasmid. This data shows that EFS is able to drive expression of human BBS1 in vitro (FIG. 2). The inventors produced and purified AAV2/8-EFS-BBS1 and injected P0 wild-type embryos with intracranial and systemic delivery. Transduction capacity of AAV2/8-EFS-BBS1 was demonstrated in both tissues, retina and brain. Specific expression of human BBS1 in the retina and brain expression was seen (FIG. 3, FIG. 4).

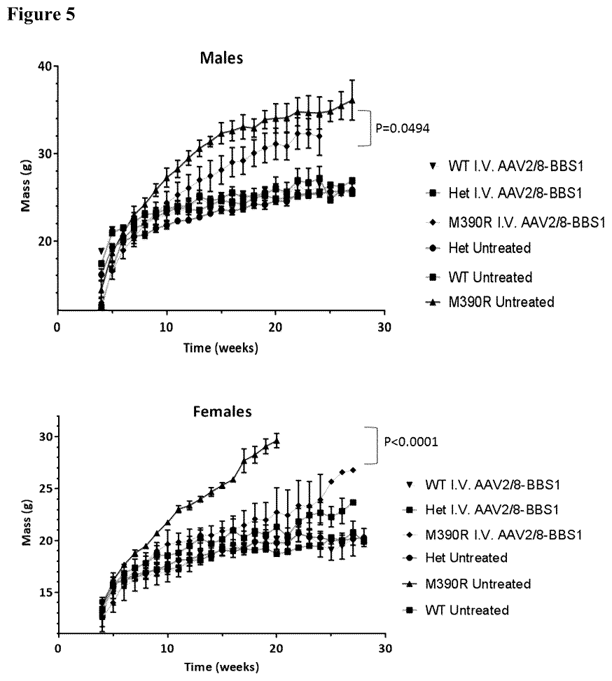

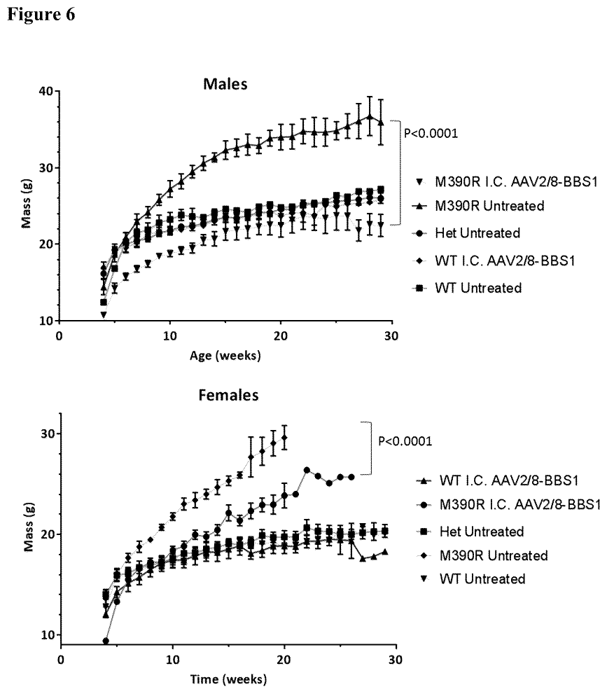

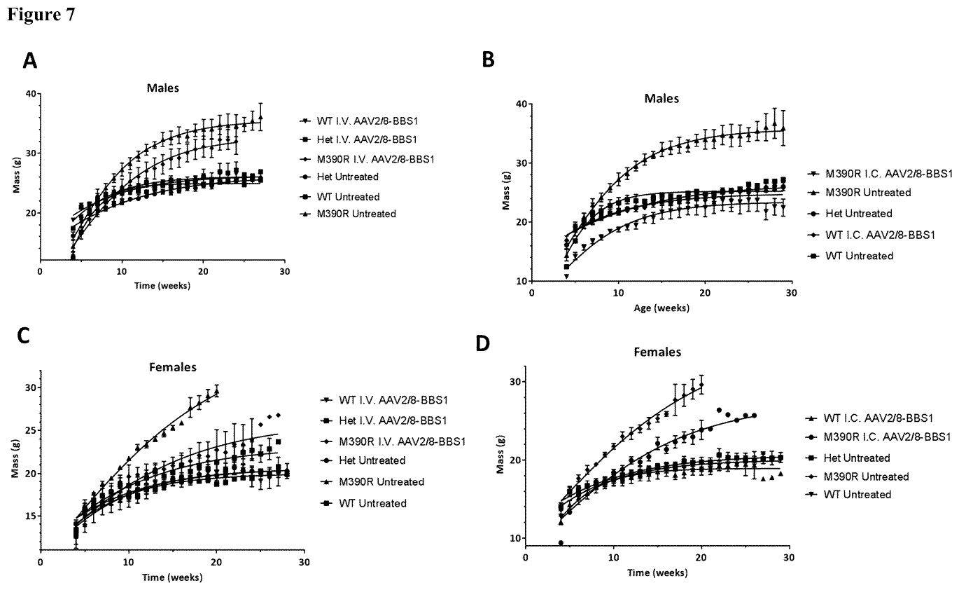

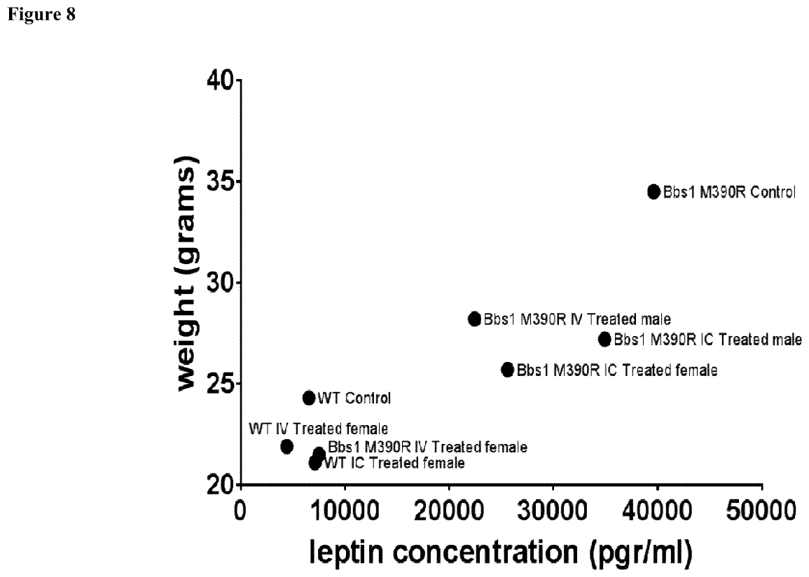

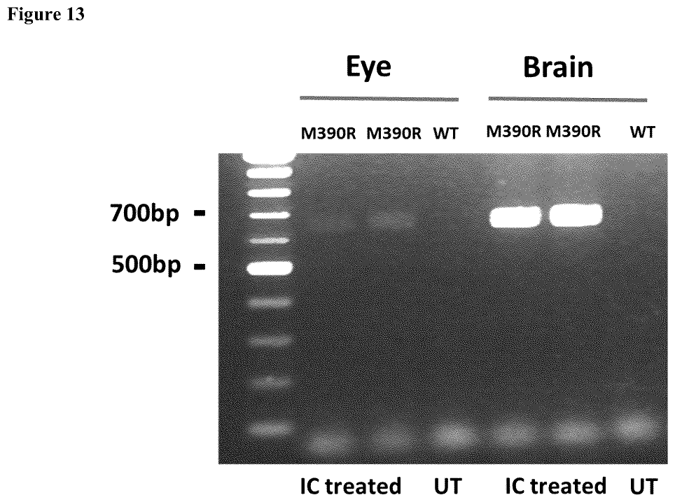

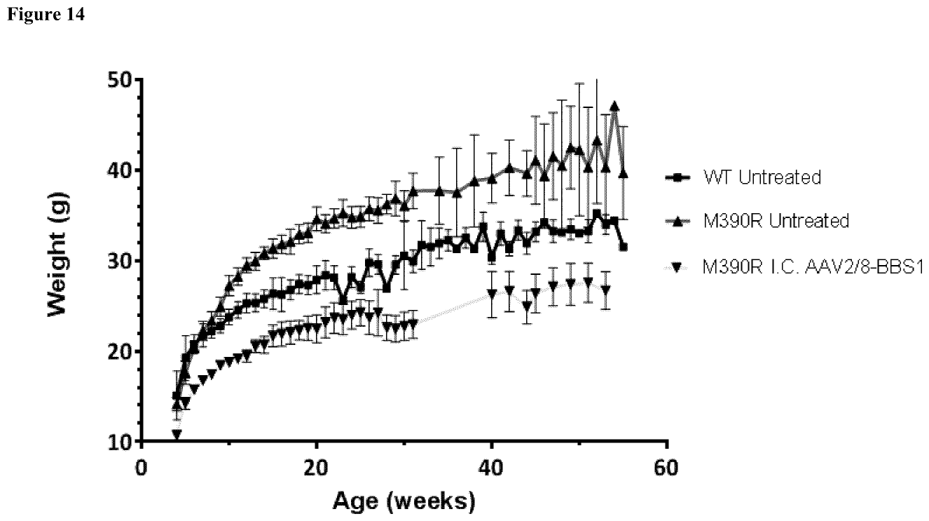

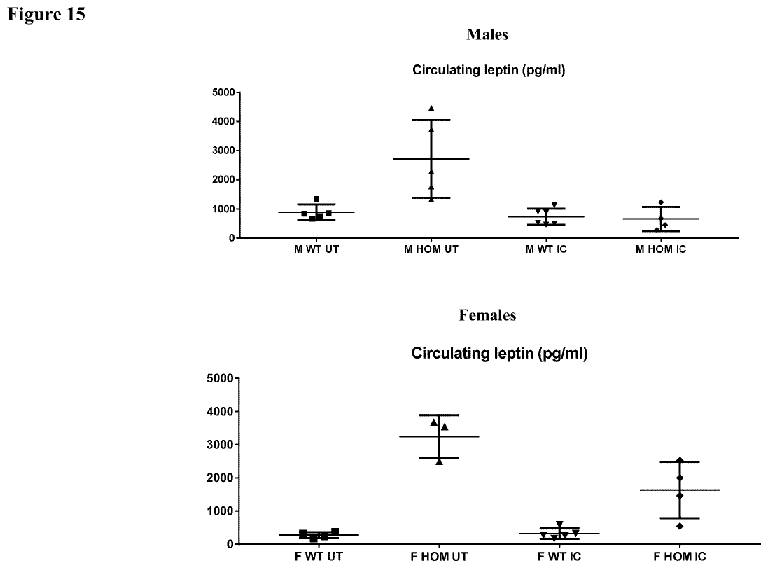

A functional study to assess the efficacy of BBS1 expression in Bbs1^{M390R/M390R} mouse model was carried out. Wild-type, heterozygous Bbs1^{M390R/30} and Bbs1^{M390R/M390R} littermates were injected at P0 with AAV2/8-EFS-BBS1. In parallel, a cohort of untreated animals from all three genotypes was kept as control. The inventors followed the cohort for 26 weeks measuring the weight of each animal every week. A significant improvement was demonstrated in body weight maintenance in both, intracranially and systemically injected Bbs1^{M390R/M390R} animals (FIGS. 5 to 7). No difference in body weight was observed between wild-type animals treated with AAV2/8-EFS-BBS1 and untreated animals. It has been shown that the levels of human BBS1 expression last for at least a year, as human BBS1 was found to be expressed in eye and retina 52 weeks after intracranial injection (FIG. 13). The improvement in regulation of body weight was also maintained for a whole year as shown in FIG. 14. Through a process of non-linear regression followed by one way ANOVA with Tukey's test statistical analysis, it was statistically proved that the rescue of body weight is significant in the Bbs1^{M390R/M390R} animals treated with AAV2/8-EFS-BBS1 compared with untreated ones (FIG. 6). Levels of leptin in serum were analysed. There was a recovery to normal levels of leptin in Bbs1^{M390R/M390R} animals treated intravenously and intracranially with AAV2/8-EFS-BBS1 (FIG. 8). The levels of normal leptin were maintained for 52 weeks for both males and females prenatally treated with AAV2/8-EFS-BBS1 (FIG. 15).

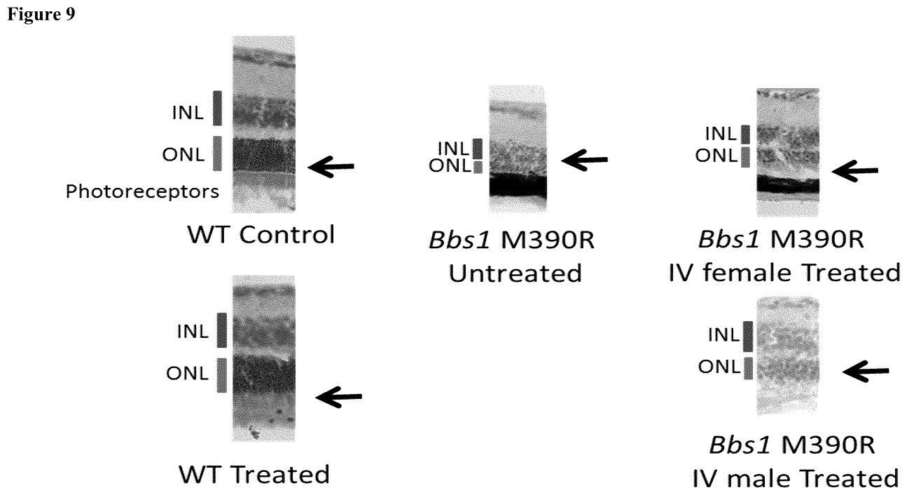

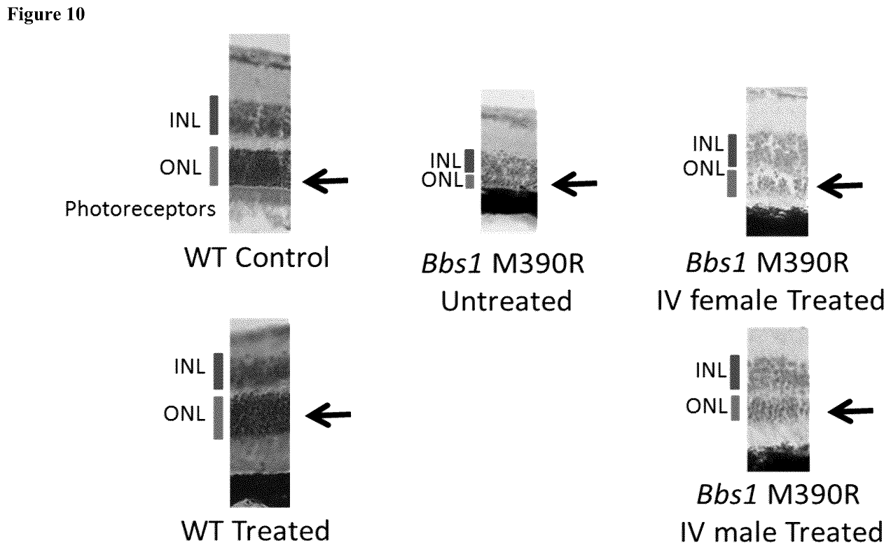

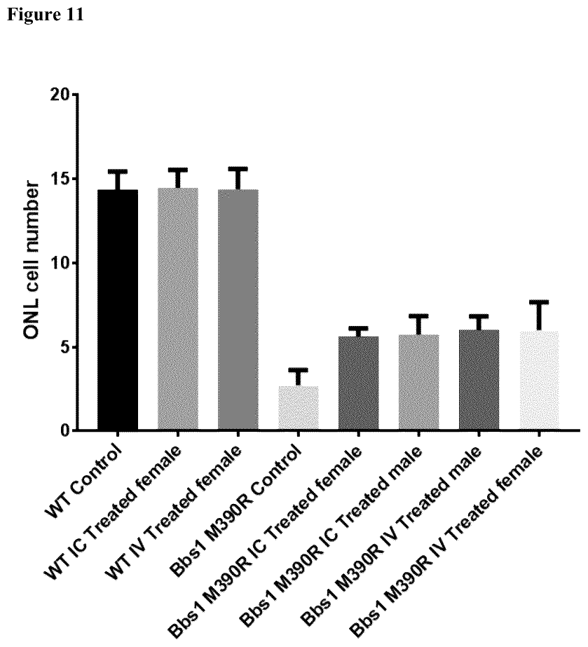

Bbs1^{M390R/M390R} animals treated at P0 with AAV2/8-EFS-BBS1 also showed an attenuation of loss in the number of outer nuclear cells ONL. The attenuation was demonstrated by quantifying the number of nuclei of surviving photoreceptors, present in the retina of treated animals at 6 months compared to untreated Bbs1^{M390R/M390R} animals. This effect was observed in both the intravenous and intracranially treated groups (FIGS. 9, 10 and 11).

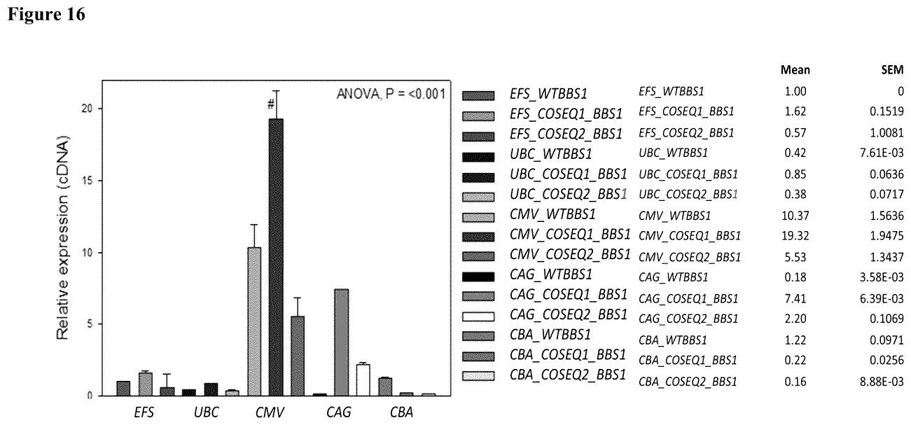

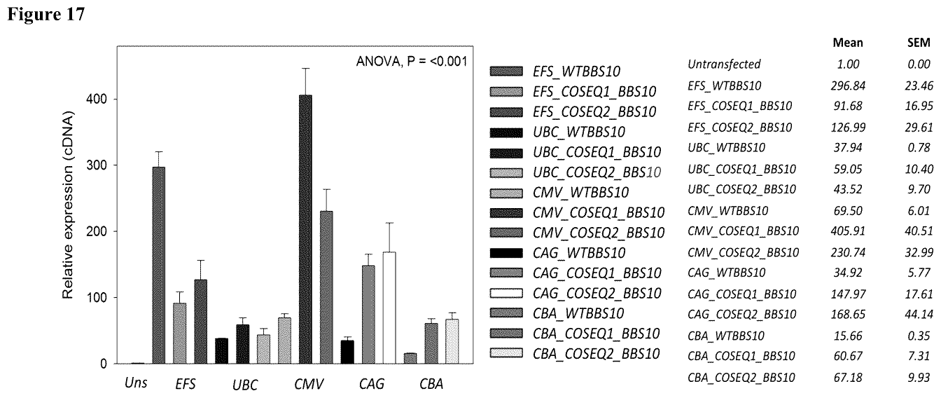

With the novel codon optimised sequences, researchers showed the relative expression of BBS1 mRNA was improved after BBS1 construct transfections (see FIG. 16). CMV promoter showed the highest levels of expression, followed by the CAG promoter expression. BBS1 codon optimised sequences, COSEQ1-BBS1 and COSEQ2-BBS1, have a much better expression than the wild-type human BBS1 cDNA. The constructs with better expression are CMV-COSEQ1-BBS1 with an increase of 19-fold, CMV-WTBBS1 with an increase of 10-fold and CAG-COSEQ1-BBS1 with an increase of 7-fold. All expression is normalised relative to EFS-WTBBS1 expression. With the novel BBS10 constructs, transfections also showed an increase in human BBS10 expression and in most of the promoter-BBS10 sequence combinations the new codon optimised BBS10 sequences deliver better yields of BBS10 RNA (FIG. 17).

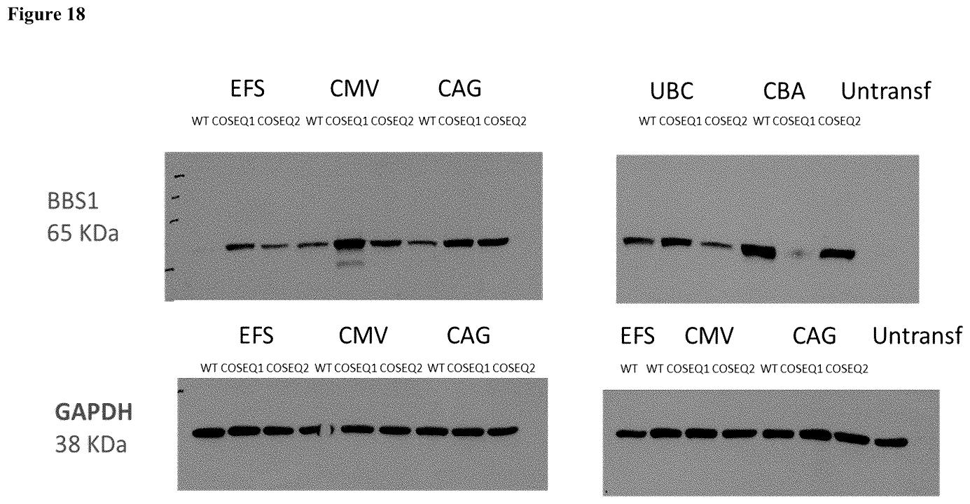

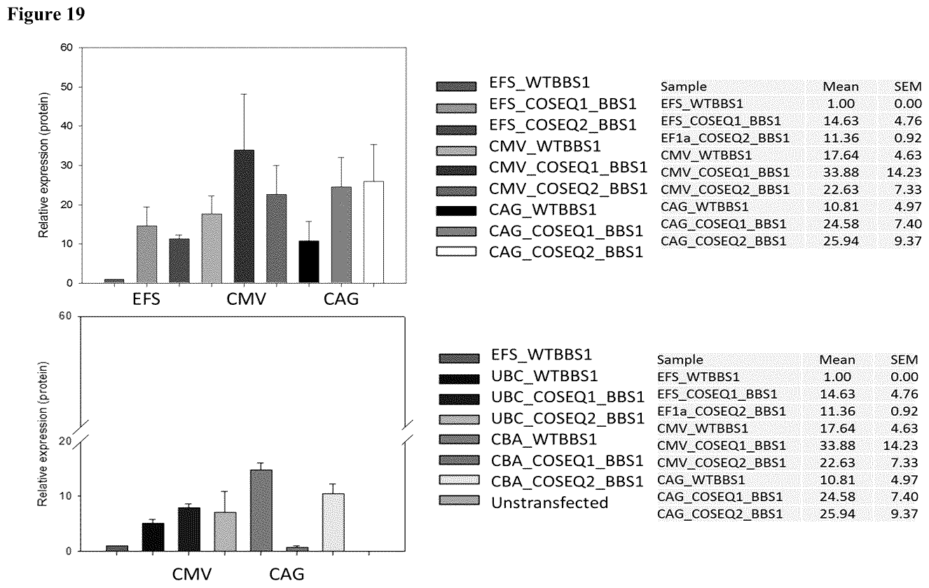

Western blots of protein extractions from all BBS1 constructs transfections showed an increase in protein expression for all BBS1 constructs. Specific bands for BBS1 (65 kDa) and for GAPDH (38 kDa) (see FIG. 18) were detected. Analysis and normalisation of the amount of protein was performed as shown in FIG. 19.

Analysis of gels show how new codon optimised sequences, COSEQ1-BBS1 and COSEQ1-BBS2, are able to express BBS1 better than wild-type BBS1, whatever the promoter that is used to drive the expression. The highest expression is found with the sequence COSEQ1-BBS1, which achieves a 33 fold increase with the CMV promoter and a 24 fold increase with the CAG promoter.

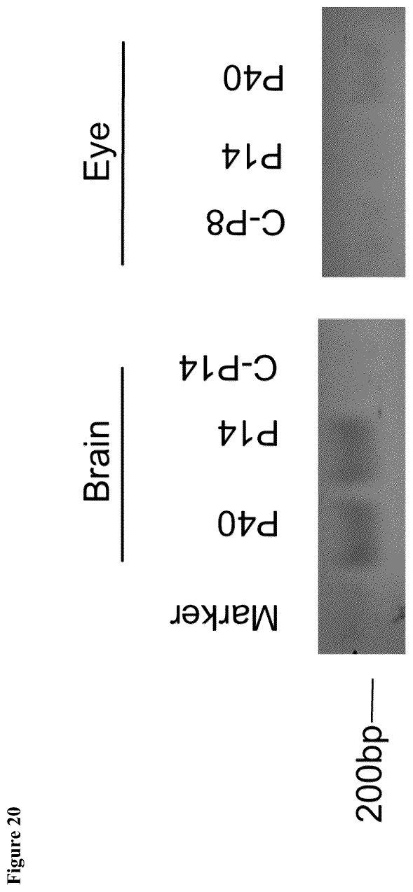

The inventors demonstrated the expression of the new codon optimised sequence COSEQ1-BBS1 in an AAV2/9-CAG-COSEQ1-BBS1 construct in the brain and the eye 40 days after transduction of the vector (see FIG. 20). To check the specificity of the band, the band was cut, cleaned and Sanger sequenced, observing the correct sequence for COSEQ1-BBS1.

### Discussion

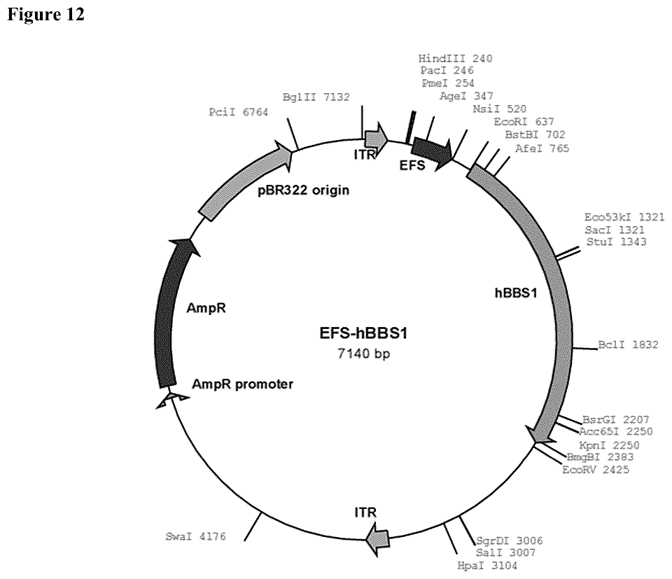

These results demonstrate that the inventors were able to deliver the human functional BBS1 gene that expresses the wild-type BBS1 protein, to multiple affected tissues with a single administration of a gene therapy vector. The increased expression of WT Bbs1 in the CNS was followed by the recovery of the function hypothalamic leptin regulation shown by the reduction in weight and a reduction of circulating leptin. Similarly, expression of human BBS1 in the eye was followed by an attenuation of the retinal degeneration six months after the systemic delivery (FIGS. 3-15).

The multi-tissue nature of most ciliopathies makes it challenging to treat some or all affected different organs with a single treatment. Even in the event a gene therapy is currently developed to treat a specific organ, it will only be useful for that specific phenotype and will not treat the condition more generally.

All ciliopathies have the same organs affected with different degrees of severity (see review N Engl J Med 2011; 364:1533-1543 Apr. 21, 2011). From all of them, BBS is one of the ciliopathies with more organs directly affected by mutations in BBS genes. The inventors have proven that gene therapy is able to target multiple affected tissues with a single vector dose. Therefore, the invention will be able to target specific ciliopathy genes in affected organs to restore function with a single administration.

Even in cases where the ciliopathy disorder is mainly affecting a single organ, such in the case of some ciliary retinal disorders, the intravenous, intracranial, and/or intravenous and intracranial administration will be more effective and risk-free than the actual techniques of subretinal therapies.

### Treatment of Other Ciliopathies

The experiments described above show that systemic expression of a protein to replace the function of the mutated gene responsible for the ciliopathy, in this case Bardet-Biedl Syndrome, is an effective way to treat some or all the organs affected by the ciliopathy. Therefore, this is a more effective way of treating the ciliopathy than previous attempts. All ciliopathies are part of a similar spectrum of disorders that affect one way or another the cilia function or structure. The phenotypical output of that relationship is that the same gene has been found to be causative for more than one ciliopathy. Common shared genes in different ciliopathies can be found, for example MKKS BBS6 is associated with Bardet-Biedl Syndrome and McKusick-Kaufman syndrome. The fact of sharing phenotypical expression, meaning the same organs are affected, and genetic homogeneity, the same gene involved in more than one ciliopathy, make this gene delivery and expression invention an unique approach to treat many ciliopathies. Therefore, this approach is not just limited to Bardet-Biedl Syndrome and it is applicable to many ciliopathies. Moreover, all ciliopathies are caused by mutations in a single gene therefore, the systemic expression of the appropriate non-mutated gene allows the pathologies associated with the ciliopathy to be ameliorated throughout the body.

As demonstrated above, Bardet-Biedl Syndrome can be treated using this gene therapy approach. The table below shows a number of genes in which mutations can occur to cause the phenotypical pathologies associated with Bardet-Biedl Syndrome. Therefore, using a gene therapy vector as described above which contains the appropriate gene to express the wild-type non-mutated protein can treat Bardet-Biedl Syndrome.

In addition, some of the genes which are associated with Bardet-Biedl Syndrome have also been associated with other related ciliopathies. As a result, the approach described above with the appropriate gene can also be used to treat other ciliopathies, such as Joubert syndrome, Meckel-Gruber syndrome, Nephronophthisis, Senior-Loken syndrome, McKusick-Kaufman syndrome and Leber's congenital amaurosis. For example, McKusick-Kaufman syndrome is caused by a mutation in the MKKS BBS6 gene. Therefore, a vector which provides expression of the MKKS BBS6 gene so that the wild type MKKS/BBS6 protein is expressed can be used to treat or ameliorate McKusick-Kaufman syndrome as well as Bardet-Biedl syndrome. This also applies to the various other ciliopathies referred to in the table below.

| Condition | Gene(s) |
|---|---|
| Bardet-Biedl syndrome | BBS1, BBS2, BBS3/ARL6, BBS4, BBS5, |
| | BBS6/MKKS, BBS7, BBS8, BBS9, BBS10, |
| | BBS11/TRIM32, BBS12, BBS13/MKS1, |
| | BBS14/CEP290, BBS15/C2ORF86, BBS16/ |
| | SDCCAG8, BBS17/LZTFL1, BBS18/BBIP1, |
| | BBS19/IFT27, BBS20/IFT74, BBS21/ |
| | C8ORF3. |
| Joubert syndrome | BBS14/CEP290 |
| Meckel-Gruber syndrome | BBS13/MKS1 |
| Nephronophthisis | BBS14/CEP290 |
| Senior-Loken syndrome | BBS14/CEP290 |
| McKusick-Kaufman | MKKS/BBS6 |
| syndrome | |
| Leber's congenital | BBS14/CEP290 |
| amaurosis | |

### SEQUENCES

SEQ ID NO. 1—Human Bardet-Biedl syndrome 1 (BBS1) nucleotide sequence (WT), cDNA (NM_024649.4)

SEQ ID NO. 2—Human Bardet-Biedl syndrome 10 (BBS10) nucleotide sequence (WT), cDNA (NM_024685.3)

SEQ ID NO. 3—Short elongation factor (EFS) promoter sequence

SEQ ID NO. 4—CAG promoter sequence

SEQ ID NO. 5—Ubiquitin C (UBC) promoter sequence

SEQ ID NO. 6—Cytomegalovirus (CMV) immediate-early promoter sequence

SEQ ID NO. 7—Phosphoglycerate kinase (PGK) promoter sequence

SEQ ID NO. 8—Chicken beta actin (CBA) promoter sequence

SEQ ID NO. 9—Human BBS1 full protein sequence (Q8NFJ9)

SEQ ID NO. 10—Human BBS10 full protein sequence (Q8TAM1)

SEQ ID NO. 11—Codon optimised nucleotide sequence encoding human BBS1 protein (referred to as COSEQ1-BBS1)

SEQ ID NO. 12—Codon optimised nucleotide sequence encoding human BBS1 protein (referred to as COSEQ2-BBS1)

SEQ ID NO. 13—Codon optimised nucleotide sequence encoding human BBS10 protein (referred to as COSEQ1-BBS10)

SEQ ID NO. 14—Codon optimised nucleotide sequence encoding human BBS10 protein (referred to as COSEQ2-BBS10)

SEQ ID NO 15—Construct comprising EFS promoter (nt 41-272) and wild type BBS1 nucleotide sequence (nt 1238-3019) (referred to as EFS-WTBBS1)

SEQ ID NO 16—Construct comprising EFS promoter (nt 41-272) and COSEQ1-BBS1 nucleotide sequence (nt 1243-3024) (referred to as EFS-COSEQ1-BBS1)

SEQ ID NO 17—Construct comprising EFS promoter (nt 41-272) and COSEQ2-BBS1 nucleotide sequence (nt 1243-3024) (referred to as EFS-COSEQ2-BBS1)

SEQ ID NO 18—Construct comprising UBC promoter (nt 29-1198) and wild type BBS1 nucleotide sequence (nt 1281-3062) (referred to as UBC-WTBBS1)

SEQ ID NO 19—Construct comprising UBC promoter (nt 29-1198) and COSEQ1-BBS1 nucleotide sequence (nt 1285-3066) (referred to as UBC-COSEQ11BBS1)

SEQ ID NO 20—Construct comprising UBC promoter (nt 29-1198) and COSEQ2-BBS1 nucleotide sequence (nt 1285-3066) (referred to as UBC-COSEQ2-BBS1)

SEQ ID NO 21—Construct comprising CMV promoter (nt 367-570) and wild type BBS1 nucleotide sequence (nt 626-2407) (referred to as CMV-WTBBS1)

SEQ ID NO 22—Construct comprising CMV promoter (nt 367-570) and COSEQ1-BBS1 nucleotide sequence (nt 630-2411) (referred to as CMV-COSEQ1-BBS1)

SEQ ID NO 23—Construct comprising CMV promoter (nt 367-570) and COSEQ2-BBS1 nucleotide sequence (nt 630-2411) (referred to as CMV-COSEQ2-BBS1)

SEQ ID NO 24—Construct comprising CBA promoter (nt 42-319) and wild type BBS1 nucleotide sequence (nt 469-2250) (referred to as CBA-WTBBS1)

SEQ ID NO 25—Construct comprising CBA promoter (nt 42-319) and COSEQ1-BBS1 nucleotide sequence (nt 473-2254) (referred to as CBA-COSEQ1-BBS1)

SEQ ID NO 26—Construct comprising CBA promoter (nt 42-319) and COSEQ2-BBS1 nucleotide sequence (nt 473-2254) (referred to as CBA-COSEQ2-BBS1)

SEQ ID NO 27—Construct comprising CAG promoter (nt 35-562) and wild type BBS1 nucleotide sequence (nt 712-2493) (referred to as CAG-WTBBS1)

SEQ ID NO 28—Construct comprising CAG promoter (nt 35-562) and COSEQ1-BBS1 nucleotide sequence (nt 716-2497) (referred to as CAG-COSEQ1-BBS1)

SEQ ID NO 29—Construct comprising CAG promoter (nt 35-562) and COSEQ2-BBS1 nucleotide sequence (nt 716-2497) (referred to as CAG-COSEQ2-BBS1)

SEQ ID NO 30—Construct comprising EFS promoter (nt 41-272) and wild type BBS10 nucleotide sequence (nt 1243-3414) (referred to as EFS-WTBBS10)

SEQ ID NO 31—Construct comprising EFS promoter (nt 41-272) and COSEQ1-BBS10 nucleotide sequence (nt 1243-3414) (referred to as EFS-COSEQ1-BBS10)

SEQ ID NO 32—Construct comprising EFS promoter (nt 41-272) and COSEQ2-BBS10 nucleotide sequence (nt 1243-3414) (referred to as EFS-COSEQ2-BBS10)

SEQ ID NO 33—Construct comprising UBC promoter (nt 29-1198) and wild type BBS10 nucleotide sequence (nt 1285-3456) (referred to as UBC-WTBBS10)

SEQ ID NO 34—Construct comprising UBC promoter (nt 29-1198) and COSEQ1-BBS10 nucleotide sequence (nt 1285-3456) (referred to as UBC-COSEQ1BBS10)

SEQ ID NO 35—Construct comprising UBC promoter (nt 29-1198) and COSEQ2-BBS10 nucleotide sequence (nt 1285-3456) (referred to as UBC-COSEQ2-BBS10)

SEQ ID NO 36—Construct comprising CMV promoter (nt 367-570) and wild type BBS10 nucleotide sequence (nt 630-2801) (referred to as CMV-WTBBS10) SEQ ID NO 37—Construct comprising CMV promoter (nt 367-570) and COSEQ1-BBS10 nucleotide sequence (nt 630-2801) (referred to as CMV-COSEQ1-BBS10)

SEQ ID NO 38—Construct comprising CMV promoter (nt 367-570) and COSEQ2-BBS10 nucleotide sequence (nt 630-2801) (referred to as CMV-COSEQ2-BBS10)

SEQ ID NO 39—Construct comprising CBA promoter (nt 42-319) and wild type BBS10 nucleotide sequence (nt 473-2644) (referred to as CBA-WTBBS10)

SEQ ID NO 40—Construct comprising CBA promoter (nt 42-319) and COSEQ1-BBS10 nucleotide sequence (nt 473-2644) (referred to as CBA-COSEQ1-BBS10)

SEQ ID NO 41—Construct comprising CBA promoter (nt 42-319) and COSEQ2-BBS10 nucleotide sequence (nt 473-2644) (referred to as CBA-COSEQ2-BBS10)

SEQ ID NO 42—Construct comprising CAG promoter (nt 35-562) and wild type BBS10 nucleotide sequence (nt 716-2887) (referred to as CAG-WTBBS10)

SEQ ID NO 43—Construct comprising CAG promoter (nt 35-562) and COSEQ1-BBS10 nucleotide sequence (nt 716-2887) (referred to as CAG-COSEQ1-BBS10)

SEQ ID NO 44—Construct comprising CAG promoter (nt 35-562) and COSEQ2-BBS10 nucleotide sequence (nt 716-2887) (referred to as CAG-COSEQ2-BBS10)

SEQ ID NO. 45—Alternative CMV promoter sequence

SEQ ID NO. 46—Alternative short elongation factor (EFS) promoter sequence

SEQ ID NO. 47—Alternative CAG promoter sequence

SEQ ID NO. 48—Alternative ubiquitin C (UBC) promoter sequence

SEQ ID NO. 49—Alternative chicken beta actin (CBA) promoter sequence

1. A vector for treating a ciliopathy, wherein the vector comprises a promoter operably linked to a ciliopathy gene, wherein the vector can provide transduction of the ciliopathy gene into multiple organs, wherein the promoter is a ubiquitous promoter which can provide expression of the ciliopathy gene in the transduced organs, and wherein the ciliopathy gene encodes a functional human protein corresponding to the protein that is mutated in the ciliopathy.

2. A vector according to claim 1, wherein the ciliopathy gene encodes a functional human BBS10 protein.

3. A vector according to claim 1, wherein the ciliopathy gene has the nucleotide sequence of SEQ ID NO. 2 or has at least 70% sequence identity thereto, and encodes a functional human BBS10 protein.

4. A vector according to claim 1, wherein the ciliopathy gene encodes a functional human BBS10 protein having the protein sequence of SEQ ID NO. 10 or has 80% sequence identity thereto.

5. A vector according to claim 1, wherein the ciliopathy gene encodes a functional BBS10 protein and has the nucleotide sequence of SEQ ID NO. 13 or has at least 70% sequence identity thereto,

6. A vector according to claim 1, wherein the ciliopathy gene encodes a functional BBS10 protein and has the nucleotide sequence of SEQ ID NO. 14 or has at least 70% sequence identity thereto.

7. A vector according to claim 1, wherein the vector is an adeno-associated viral (AAV) vector or a lentiviral vector.

8. A vector according to claim 1, wherein the vector is selected from AAV8, AAV9, AAV vectors pseudotyped with the capsid proteins from AAV8 or AAV9, AAV-PHP.A, AAV-PHP.B, AAV9.47, AAV-B1, AAV8 (Y733F) or AAV2-TT.

9. A vector according to claim 1, wherein the vector is an AAV8 vector, an AAV9 vector, or an AAV vector which has been pseudotyped with the capsid proteins from AAV8 or AAV9.

10. A vector according to claim 1, wherein the promoter is selected from the short elongation factor promoter (EFS), CAG promoter, cytomegalovirus immediate-early promoter (CMV), Ubiquitin C promoter (UBC), phosphoglycerate kinase promoter (PGK) and beta-actin promoter.

11. A vector according to claim 1, wherein the promoter has a sequence selected from SEQ ID NO. 3, SEQ ID NO. 4, SEQ ID NO. 5, SEQ ID NO. 6, SEQ ID NO. 7, SEQ ID NO. 8 and SEQ ID NO. 45.

12. A vector according to claim 1, wherein the promoter is a CAG promoter.

13. A vector according to claim 1, wherein the promoter has the nucleotide sequence of SEQ ID NO. 4 or SEQ ID NO. 47.

14. A vector according to claim 1, wherein the ciliopathy is selected from Bardet-Biedl syndrome, McKusick-Kaufman syndrome, Joubert syndrome, Meckel-Gruber syndrome, nephronophthisis, Senior-Loken syndrome, Leber's congenital amaurosis.

15. A vector according to claim 1, wherein the vector is an AAV8 vector, an AAV9 vector, or an AAV vector which has been pseudotyped with the capsid proteins from AAV8 or AAV9,
wherein the promoter is a CAG promoter, and
wherein the ciliopathy gene encodes a functional human BBS10 protein.

16. A pharmaceutical composition comprising the vector according to claim 1 and one or more pharmaceutically acceptable excipients.

17. A method of treating a ciliopathy comprising administering a therapeutically effective amount of a vector according to claim 1 to a patient suffering from a ciliopathy.

18. The vector according to claim 1 for use in therapy.


SEQUENCE LISTING (49 sequences)

SEQ ID NO: 1 - DNA, 1782 bp, Homo sapiens
  source 1..1782: mol_type = other DNA
atggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctga

SEQ ID NO: 2 - DNA, 2172 bp, Homo sapiens
  source 1..2172: mol_type = other DNA
atgttaagttctatggccgctgcagggtctgtgaaggcggcgttgcaggtggccgaggtgctggaagccatcgtgagctgctgcgtggggcccgagggacggcaagttttgtgtacgaagcccactggcgaggtgcttctcagccggaatggaggccgcctcctggaggcgctacacttagagcatcccatagccaggatgatagtggactgtgtttccagtcatctcaaaaaaacaggagatggtgcaaaaacatttattatctttctttgccatttgcttagaggacttcatgcaatcacagacagagaaaaggatcctttgatgtgtgaaaacattcaaacccatggaaggcattggaaaaattgttctcggtggaaatttatttcccaggctctcctaacgtttcagacacaaatattagacggtattatggaccagtacctaagtagacactttttgtctatcttttcgtctgctaaagagagaacattgtgtaggagctctttagagttgctcttagaagcatacttttgtggaagagtgggaagaaataatcataaatttatttcacagttgatgtgtgactactttttcaagtgtatgacttgtaaaagtgggattggtgtatttgagttagtggatgaccattttgtagagttgaatgttggtgtcactggccttcctgtttcagattccaggatcatagctggtcttgtgcttcagaaagatttttctgtgtaccgcccagcagatggtgacatgcgaatggtgatagtaacagaaaccattcagcctcttttttccacttctggatcagagtttattctaaattcagaagcacagtttcagacatctcaattttggattatggaaaagacaaaagcaataatgaaacatctacatagtcagaatgtaaaattgctcatatctagtgtgaaacaaccagatttagttagttattatgcaggggtgaatggcatatcagtggttgagtgtttatcatcagaagaagtttctcttatccggaggatcattggtctttctccatttgtaccaccacaggccttttcgcagtgtgaaatacctaacactgctttggtgaaattttgtaaacctcttatccttagatccaaaagatatgttcatctaggcttgataagcacatgtgcatttataccacactctatagttctttgtggaccagtgcatggtctcattgaacaacatgaggatgctttacatggagcacttaaaatgcttcggcaattatttaaagaccttgatctaaattacatgacacaaaccaatgaccaaaatggcacttcaagtctttttatttataagaacagtggagaaagttatcaagcaccagatcctggtaatggctcaatacaaaggccttatcaggacacagttgcagagaacaaagatgcattggaaaaaactcaaacatatttaaaagtacattctaatttggtaattccagatgtagaattagaaacatatattccgtattcaacccccacactgacaccaacagatacattccaaacagttgaaacgctgacatgtttgtctttggaaagaaacaggctaactgattattatgaaccattactcaagaacaattccactgcttattcaacaaggggaaatagaatagaaatttcttacgaaaatttacaggtcacaaatattactagaaagggaagcatgttaccagtgagctgtaagttaccgaatatgggtacttcccagagttacctttcctcatctatgccagctggttgtgttttgccagtaggtggtaattttgagatcttgttacattactatcttctcaattatgccaaaaaatgccatcaatcagaagaaaccatggttagtatgataatagctaatgcacttttaggcattcccaaagtcctttataaatctaaaacaggaaagtacagctttccacatacatatataagagctgtccatgcactgcaaaccaatcaacccttggtaagcagtcagacaggtttggaatcagtaatgggtaaataccagctactaacttcagttcttcagtgtttgacaaaaatattaaccattgacatggtaatcactgttaagagacaccctcagaaagttcacaatcaagattcagaagatgaactataa

SEQ ID NO: 3 - DNA, 232 bp, Homo sapiens
  source 1..232: mol_type = other DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 4 - DNA, 584 bp, synthetic construct
  misc_feature 1..584: note = CAG promoter sequence
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 5 - DNA, 1177 bp, Homo sapiens
  source 1..1177: mol_type = other DNA
ggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 6 - DNA, 511 bp, Human cytomegalovirus
  source 1..511: mol_type = other DNA
gatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggtcattattgacgtcaatgggcgggggtcgttgggcggtcagccaggcgggccatttaccgtaagttatgtaacggg

SEQ ID NO: 7 - DNA, 511 bp, Mus musculus
  source 1..511: mol_type = other DNA
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 8 - DNA, 260 bp, Gallus gallus
  source 1..260: mol_type = other DNA
cacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgg

SEQ ID NO: 9 - protein, 593 aa, Homo sapiens
MAAASSSDSDACGAESNEANSKWLDAHYDPMANIHTFSACLALADLHGDGEYKLVVGDLGPGGQQPRLKVLKGPLVMTESPLPALPAAAATFLMEQHEPRTPALALASGPCVYVYKNLRPYFKFSLPQLPPNPLEQDLWNQAKEDRIDPLTLKEMLESIRETAEEPLSIQSLRFLQLELSEMEAFVNQHKSNSIKRQTVITTMTTLKKNLADEDAVSCLVLGTENKELLVLDPEAFTILAKMSLPSVPVFLEVSGQFDVEFRLAAACRNGNIYILRRDSKHPKYCIELSAQPVGLIRVHKVLVVGSTQDSLHGFTHKGKKLWTVQMPAAILTMNLLEQHSRGLQAVMAGLANGEVRIYRDKALLNVIHTPDAVTSLCFGRYGREDNTLIMTTRGGGLIIKILKRTAVFVEGGSEVGPPPAQAMKLNVPRKTRLYVDQTLREREAGTAMHRAFQTDLYLLRLRAARAYLQALESSLSPLSTTAREPLKLHAVVQGLGPTFKLTLHLQNTSTTRPVLGLLVCFLYNEALYSLPRAFFKVPLLVPGLNYPLETFVESLSNKGISDIIKVLVLREGQSAPLLSAHVNMPGSEGLAAA

SEQ ID NO: 10 - protein, 723 aa, Homo sapiens
MLSSMAAAGSVKAALQVAEVLEAIVSCCVGPEGRQVLCTKPTGEVLLSRNGGRLLEALHLEHPIARMIVDCVSSHLKKTGDGAKTFIIFLCHLLRGLHAITDREKDPLMCENIQTHGRHWKNCSRWKFISQALLTFQTQILDGIMDQYLSRHFLSIFSSAKERTLCRSSLELLLEAYFCGRVGRNNHKFISQLMCDYFFKCMTCKSGIGVFELVDDHFVELNVGVTGLPVSDSRIIAGLVLQKDFSVYRPADGDMRMVIVTETIQPLFSTSGSEFILNSEAQFQTSQFWIMEKTKAIMKHLHSQNVKLLISSVKQPDLVSYYAGVNGISVVECLSSEEVSLIRRIIGLSPFVPPQAFSQCEIPNTALVKFCKPLILRSKRYVHLGLISTCAFIPHSIVLCGPVHGLIEQHEDALHGALKMLRQLFKDLDLNYMTQTNDQNGTSSLFIYKNSGESYQAPDPGNGSIQRPYQDTVAENKDALEKTQTYLKVHSNLVIPDVELETYIPYSTPTLTPTDTFQTVETLTCLSLERNRLTDYYEPLLKNNSTAYSTRGNRIEISYENLQVTNITRKGSMLPVSCKLPNMGTSQSYLSSSMPAGCVLPVGGNFEILLHYYLLNYAKKCHQSEETMVSMIIANALLGIPKVLYKSKTGKYSFPHTYIRAVHALQTNQPLVSSQTGLESVMGKYQLLTSVLQCLTKILTIDMVITVKRHPQKVHNQDSEDEL

SEQ ID NO: 11 - DNA, 1782 bp, synthetic construct
  misc_feature 1..1782: note = Codon optimised nucleotide sequence encoding human BBS1 protein (referred to as COSEQ1-BBS1)
  source 1..1782: mol_type = other DNA
atggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaa

SEQ ID NO: 12 - DNA, 1782 bp, synthetic construct
  misc_feature 1..1782: note = Codon optimised nucleotide sequence encoding human BBS1 protein (referred to as COSEQ2-BBS1)
  source 1..1782: mol_type = other DNA
atggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctga

SEQ ID NO: 13 - DNA, 2172 bp, synthetic construct
  misc_feature 1..2172: note = Codon optimised nucleotide sequence encoding human BBS10 protein (referred to as COSEQ1-BBS10)
  source 1..2172: mol_type = other DNA
atgctgtctagcatggctgccgctggatctgtgaaagctgctctgcaggttgccgaggtgctggaagccatcgtgtcttgttgtgtgggacctgagggcagacaggtgctgtgtacaaagcctacaggcgaagtgctgctgagcagaaatggcggcagactgctcgaagccctgcacctggaacaccctatcgccagaatgatcgtggactgcgtgtccagccacctgaagaaaacaggcgacggcgccaagaccttcatcatctttctgtgccatctgctgcggggcctgcacgccatcaccgatagagaaaaggaccctctgatgtgcgagaacatccagacacacggccggcactggaagaactgctcccggtggaagtttatcagccaggctctgctgacctttcagacccagatcctggacggcatcatggaccagtacctgagcagacacttcctgagcatcttcagcagcgccaaagaacggaccctgtgcagaagctctctggaactgctgctcgaggcctacttttgcggcagagtgggcagaaacaaccacaagttcatctcccagctgatgtgtgactacttcttcaagtgcatgacctgcaagagcggcatcggcgtgttcgagctggtggacgatcacttcgtggaactgaatgtgggcgtgaccggcctgcctgtgtccgatagcagaattattgccggcctggtgctgcagaaagacttcagcgtgtacagacccgccgacggcgacatgagaatggtcatcgtgaccgagacaatccagcctctgttcagcacaagcggcagcgagttcatcctgaacagcgaggcccagtttcagaccagccagttctggatcatggaaaagaccaaggccatcatgaagcacctccacagccagaacgtgaagctgctgatctccagcgtgaagcagcccgacctggtgtcttattatgccggcgtgaacggcatcagcgtggtggaatgtctgagcagcgaagaggtgtccctgatcagacggatcatcggactgagcccctttgtgcctcctcaagcctttagccagtgcgagatccctaacacagccctggtcaagttctgcaagcccctgatcctgcggagcaagagatatgtgcacctgggcctgatcagcacatgcgccttcattcctcactccatcgtgctgtgtggacctgtgcacggactgattgagcagcacgaagatgcactgcacggcgccctgaaaatgctgagacagctgttcaaggacctggacctgaactacatgacccagaccaacgaccagaacggcaccagcagcctgttcatctacaagaacagcggcgagagctatcaggccccagatccaggcaatggcagcatccagaggccttaccaggataccgtggccgagaacaaggacgccctggaaaaaacccagacctacctgaaggtgcacagcaacctggtcatccccgatgtggaactggaaacctacattccctacagcacccctacactgacccctaccgataccttccagaccgtggaaaccctgacctgtctgagcctggaacggaacagactgaccgactactacgagcccctgctgaaaaacaacagcaccgcctatagcacccggggcaacagaatcgagatcagctacgagaacctgcaagtgaccaacatcacccggaagggctccatgctgccagtgtcctgcaagctgcctaatatgggcaccagccagagctacctgtcctcttctatgcctgccggatgtgtgctgcctgtcggcggcaattttgagatcctgctgcactactacctgctgaactacgccaagaagtgccaccagagcgaagagacaatggtgtccatgattatcgccaacgctctgctgggcatccccaaggtgctgtacaagagcaagaccggcaagtacagcttccctcacacctacattagagccgtgcacgccctgcagaccaatcagccactggtttctagccagacaggcctggaaagcgtgatgggaaagtaccagctgctgaccagcgtgctgcagtgcctgaccaagatcctgaccatcgacatggtcatcaccgtgaagcggcaccctcagaaagtgcacaaccaggacagcgaggacgagctgtag

SEQ ID NO: 14 - DNA, 2172 bp, synthetic construct
  misc_feature 1..2172: note = Codon optimised nucleotide sequence encoding human BBS10 protein (referred to as COSEQ2-BBS10)
  source 1..2172: mol_type = other DNA
atgctgagctccatggctgccgccggatctgtgaaagccgccctgcaggtggcagaggtgctggaggcaatcgtgagctgttgcgtgggacctgagggccggcaggtgctgtgcacaaagccaaccggcgaggtgctgctgtctagaaatggcggccggctgctggaggccctgcacctggagcacccaattgcaagaatgatcgtggactgcgtgtctagccacctgaagaagacaggcgatggcgccaagaccttcatcatcttcctgtgccacctgctgaggggcctgcacgccatcacagaccgcgagaaggatcctctgatgtgcgagaacatccagacccacggcaggcactggaagaattgttcccgctggaagttcatctctcaggccctgctgacatttcagacccagatcctggacggcatcatggatcagtatctgagcaggcacttcctgtccatcttttcctctgccaaggagcggaccctgtgcagaagctccctggagctgctgctggaggcctacttctgtggccgggtgggcagaaacaatcacaagtttatcagccagctgatgtgcgactatttctttaagtgcatgacctgtaagtccggcatcggcgtgttcgagctggtggacgatcactttgtggagctgaacgtgggagtgacaggcctgcccgtgtccgactctcgcatcatcgccggcctggtgctgcagaaggatttctccgtgtaccggcctgccgacggcgatatgagaatggtcatcgtgaccgagacaatccagccactgttcagcacctccggctctgagttcatcctgaacagcgaggcccagttccagacatctcagttttggatcatggagaagaccaaggccatcatgaagcacctgcacagccagaacgtgaagctgctgatctctagcgtgaagcagccagacctggtgtcttactatgccggcgtgaatggcatcagcgtggtggagtgtctgtcctctgaggaggtgtccctgatccggagaatcattggcctgtctcccttcgtgccccctcaggcctttagccagtgcgagatccccaacacagccctggtgaagttctgtaagcctctgatcctgaggtccaagcggtacgtgcacctgggcctgatcagcacctgcgcctttatcccacactctatcgtgctgtgcggacctgtgcacggcctgattgagcagcacgaggatgcactgcacggcgccctgaagatgctgaggcagctgttcaaggacctggatctgaattacatgacccagacaaacgaccagaatggcacaagctccctgtttatctacaagaactctggcgagagctatcaggccccagatcccggcaatggcagcattcagcgcccctaccaggacacagtggcagagaacaaggatgccctggagaagacccagacatatctgaaggtgcactccaacctggtcatccctgacgtggagctggagacatacatcccttattctaccccaacactgacccccacagataccttccagacagtggagacactgacctgcctgtccctggagaggaaccgcctgaccgactactatgagcccctgctgaagaacaattccacagcctactctacccggggcaatagaatcgagatcagctatgagaacctgcaggtgacaaatatcaccagaaagggctctatgctgcctgtgagctgcaagctgccaaacatgggcaccagccagtcctacctgtctagctccatgcctgcaggatgcgtgctgcctgtgggcggcaacttcgagatcctgctgcactactatctgctgaattatgccaagaagtgccaccagagcgaggagacaatggtgtccatgatcatcgccaacgccctgctgggcatcccaaaggtgctgtacaagtctaagaccggcaagtacagctttccccacacctatatccgggccgtgcacgccctgcagacaaatcagcctctggtgtctagccagaccggcctggagtccgtgatgggcaagtaccagctgctgacatctgtgctgcagtgtctgacaaagatcctgaccatcgatatggtcatcaccgtgaagagacacccacagaaggtgcacaatcaggacagcgaggatgagctgtaa

SEQ ID NO: 15 - DNA, 3030 bp, synthetic construct
  misc_feature 1..3030: note = Construct comprising EFS promoter and wild type BBS1 nucleotide sequence (referred to as EFS-WTBBS1)
  source 1..3030: mol_type = other DNA
actagtgggcagatctcgatcgagttgggccccagagcttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattactccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccatgagcttcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgac

SEQ ID NO: 16 - DNA, 3035 bp, synthetic construct
  misc_feature 1..3035: note = Construct comprising EFS promoter and COSEQ1-BBS1 nucleotide sequence (referred to as EFS-COSEQ1-BBS1)
  source 1..3035: mol_type = other DNA
actagtgggcagatctcgatcgagttgggccccagagcttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccatgagcttcgaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcgccgtcgac

SEQ ID NO: 17 - DNA, 3038 bp, synthetic construct
  misc_feature 1..3038: note = Construct comprising EFS promoter and COSEQ2-BBS1 nucleotide sequence (referred to as EFS-COSEQ2-BBS1)
  source 1..3038: mol_type = other DNA
actagtgggcagatctcgatcgagttgggccccagagcttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccatgagcttcgaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 18 - DNA, 3073 bp, synthetic construct
  misc_feature 1..3073: note = Construct comprising UBC promoter and wild type BBS1 nucleotide sequence (referred to as UBC-WTBBS1)
  source 1..3073: mol_type = other DNA
actagtaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgagctcaagcttcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgac

SEQ ID NO: 19 - DNA, 3080 bp, synthetic construct
  misc_feature 1..3080: note = Construct comprising UBC promoter and COSEQ1-BBS1 nucleotide sequence (referred to as UBC-COSEQ11BBS1)
  source 1..3080: mol_type = other DNA
actagtaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgagctcaagcttcgaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcggccgcgtcgac

SEQ ID NO: 20 - DNA, 3080 bp, synthetic construct
  misc_feature 1..3080: note = Construct comprising UBC promoter and COSEQ2-BBS1 nucleotide sequence (referred to as UBC-COSEQ2-BBS1)
  source 1..3080: mol_type = other DNA
actagtaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgagctcaagcttcgaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 21 - DNA, 2418 bp, synthetic construct
  misc_feature 1..2418: note = Construct comprising CMV promoter and wild type BBS1 nucleotide sequence (referred to as CMV-WTBBS1)
  source 1..2418: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgac

SEQ ID NO: 22 - DNA, 2422 bp, synthetic construct
  misc_feature 1..2422: note = Construct comprising CMV promoter and COSEQ1-BBS1 nucleotide sequence (referred to as CMV-COSEQ1-BBS1)
  source 1..2422: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcgccgtcgac

SEQ ID NO: 23 - DNA, 2425 bp, synthetic construct
  misc_feature 1..2425: note = Construct comprising CMV promoter and COSEQ2-BBS1 nucleotide sequence (referred to as CMV-COSEQ2-BBS1)
  source 1..2425: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 24 - DNA, 2261 bp, synthetic construct
  misc_feature 1..2261: note = Construct comprising CBA promoter and wild type BBS1 nucleotide sequence (referred to as CBA-WTBBS1)
  source 1..2261: mol_type = other DNA
actagtgtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgac

SEQ ID NO: 25 - DNA, 2265 bp, synthetic construct
  misc_feature 1..2265: note = Construct comprising CBA promoter and COSEQ1-BBS1 nucleotide sequence (referred to as CBA-COSEQ1-BBS1)
  source 1..2265: mol_type = other DNA
actagtgtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcgccgtcgac

SEQ ID NO: 26 - DNA, 2268 bp, synthetic construct
  misc_feature 1..2268: note = Construct comprising CBA promoter and COSEQ2-BBS1 nucleotide sequence (referred to as CBA-COSEQ2-BBS1)
  source 1..2268: mol_type = other DNA
actagtgtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 27 - DNA, 2504 bp, synthetic construct
  misc_feature 1..2504: note = Construct comprising CAG promoter and wild type BBS1 nucleotide sequence (referred to as CAG-WTBBS1)
  source 1..2504: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgac

SEQ ID NO: 28 - DNA, 2508 bp, synthetic construct
  misc_feature 1..2508: note = Construct comprising CAG promoter and COSEQ1-BBS1 nucleotide sequence (referred to as CAG-COSEQ1-BBS1)
  source 1..2508: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcgccgtcgac

SEQ ID NO: 29 - DNA, 2511 bp, synthetic construct
  misc_feature 1..2511: note = Construct comprising CAG promoter and COSEQ2-BBS1 nucleotide sequence (referred to as CAG-COSEQ2-BBS1)
  source 1..2511: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 30 - DNA, 3428 bp, synthetic construct
  misc_feature 1..3428: note = Construct comprising EFS promoter and wild type BBS10 nucleotide sequence (referred to as EFS-WTBBS10)
  source 1..3428: mol_type = other DNA
actagtgggcagatctcgatcgagttgggccccagagcttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccatgagcttcgaattcgccaccatgttaagttctatggccgctgcagggtctgtgaaggcggcgttgcaggtggccgaggtgctggaagccatcgtgagctgctgcgtggggcccgagggacggcaagttttgtgtacgaagcccactggcgaggtgcttctcagccggaatggaggccgcctcctggaggcgctacacttagagcatcccatagccaggatgatagtggactgtgtttccagtcatctcaaaaaaacaggagatggtgcaaaaacatttattatctttctttgccatttgcttagaggacttcatgcaatcacagacagagaaaaggatcctttgatgtgtgaaaacattcaaacccatggaaggcattggaaaaattgttctcggtggaaatttatttcccaggctctcctaacgtttcagacacaaatattagacggtattatggaccagtacctaagtagacactttttgtctatcttttcgtctgctaaagagagaacattgtgtaggagctctttagagttgctcttagaagcatacttttgtggaagagtgggaagaaataatcataaatttatttcacagttgatgtgtgactactttttcaagtgtatgacttgtaaaagtgggattggtgtatttgagttagtggatgaccattttgtagagttgaatgttggtgtcactggccttcctgtttcagattccaggatcatagctggtcttgtgcttcagaaagatttttctgtgtaccgcccagcagatggtgacatgcgaatggtgatagtaacagaaaccattcagcctcttttttccacttctggatcagagtttattctaaattcagaagcacagtttcagacatctcaattttggattatggaaaagacaaaagcaataatgaaacatctacatagtcagaatgtaaaattgctcatatctagtgtgaaacaaccagatttagttagttattatgcaggggtgaatggcatatcagtggttgagtgtttatcatcagaagaagtttctcttatccggaggatcattggtctttctccatttgtaccaccacaggccttttcgcagtgtgaaatacctaacactgctttggtgaaattttgtaaacctcttatccttagatccaaaagatatgttcatctaggcttgataagcacatgtgcatttataccacactctatagttctttgtggaccagtgcatggtctcattgaacaacatgaggatgctttacatggagcacttaaaatgcttcggcaattatttaaagaccttgatctaaattacatgacacaaaccaatgaccaaaatggcacttcaagtctttttatttataagaacagtggagaaagttatcaagcaccagatcctggtaatggctcaatacaaaggccttatcaggacacagttgcagagaacaaagatgcattggaaaaaactcaaacatatttaaaagtacattctaatttggtaattccagatgtagaattagaaacatatattccgtattcaacccccacactgacaccaacagatacattccaaacagttgaaacgctgacatgtttgtctttggaaagaaacaggctaactgattattatgaaccattactcaagaacaattccactgcttattcaacaaggggaaatagaatagaaatttcttacgaaaatttacaggtcacaaatattactagaaagggaagcatgttaccagtgagctgtaagttaccgaatatgggtacttcccagagttacctttcctcatctatgccagctggttgtgttttgccagtaggtggtaattttgagatcttgttacattactatcttctcaattatgccaaaaaatgccatcaatcagaagaaaccatggttagtatgataatagctaatgcacttttaggcattcccaaagtcctttataaatctaaaacaggaaagtacagctttccacatacatatataagagctgtccatgcactgcaaaccaatcaacccttggtaagcagtcagacaggtttggaatcagtaatgggtaaataccagctactaacttcagttcttcagtgtttgacaaaaatattaaccattgacatggtaatcactgttaagagacaccctcagaaagttcacaatcaagattcagaagatgaactataagcggccgcgtcgac

SEQ ID NO: 31 - DNA, 3425 bp, synthetic construct
  misc_feature 1..3425: note = Construct comprising EFS promoter and COSEQ1-BBS10 nucleotide sequence (referred to as EFS-COSEQ1-BBS10)
  source 1..3425: mol_type = other DNA
actagtgggcagatctcgatcgagttgggccccagagcttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccatgagcttcgaattcgccaccatgctgtctagcatggctgccgctggatctgtgaaagctgctctgcaggttgccgaggtgctggaagccatcgtgtcttgttgtgtgggacctgagggcagacaggtgctgtgtacaaagcctacaggcgaagtgctgctgagcagaaatggcggcagactgctcgaagccctgcacctggaacaccctatcgccagaatgatcgtggactgcgtgtccagccacctgaagaaaacaggcgacggcgccaagaccttcatcatctttctgtgccatctgctgcggggcctgcacgccatcaccgatagagaaaaggaccctctgatgtgcgagaacatccagacacacggccggcactggaagaactgctcccggtggaagtttatcagccaggctctgctgacctttcagacccagatcctggacggcatcatggaccagtacctgagcagacacttcctgagcatcttcagcagcgccaaagaacggaccctgtgcagaagctctctggaactgctgctcgaggcctacttttgcggcagagtgggcagaaacaaccacaagttcatctcccagctgatgtgtgactacttcttcaagtgcatgacctgcaagagcggcatcggcgtgttcgagctggtggacgatcacttcgtggaactgaatgtgggcgtgaccggcctgcctgtgtccgatagcagaattattgccggcctggtgctgcagaaagacttcagcgtgtacagacccgccgacggcgacatgagaatggtcatcgtgaccgagacaatccagcctctgttcagcacaagcggcagcgagttcatcctgaacagcgaggcccagtttcagaccagccagttctggatcatggaaaagaccaaggccatcatgaagcacctccacagccagaacgtgaagctgctgatctccagcgtgaagcagcccgacctggtgtcttattatgccggcgtgaacggcatcagcgtggtggaatgtctgagcagcgaagaggtgtccctgatcagacggatcatcggactgagcccctttgtgcctcctcaagcctttagccagtgcgagatccctaacacagccctggtcaagttctgcaagcccctgatcctgcggagcaagagatatgtgcacctgggcctgatcagcacatgcgccttcattcctcactccatcgtgctgtgtggacctgtgcacggactgattgagcagcacgaagatgcactgcacggcgccctgaaaatgctgagacagctgttcaaggacctggacctgaactacatgacccagaccaacgaccagaacggcaccagcagcctgttcatctacaagaacagcggcgagagctatcaggccccagatccaggcaatggcagcatccagaggccttaccaggataccgtggccgagaacaaggacgccctggaaaaaacccagacctacctgaaggtgcacagcaacctggtcatccccgatgtggaactggaaacctacattccctacagcacccctacactgacccctaccgataccttccagaccgtggaaaccctgacctgtctgagcctggaacggaacagactgaccgactactacgagcccctgctgaaaaacaacagcaccgcctatagcacccggggcaacagaatcgagatcagctacgagaacctgcaagtgaccaacatcacccggaagggctccatgctgccagtgtcctgcaagctgcctaatatgggcaccagccagagctacctgtcctcttctatgcctgccggatgtgtgctgcctgtcggcggcaattttgagatcctgctgcactactacctgctgaactacgccaagaagtgccaccagagcgaagagacaatggtgtccatgattatcgccaacgctctgctgggcatccccaaggtgctgtacaagagcaagaccggcaagtacagcttccctcacacctacattagagccgtgcacgccctgcagaccaatcagccactggtttctagccagacaggcctggaaagcgtgatgggaaagtaccagctgctgaccagcgtgctgcagtgcctgaccaagatcctgaccatcgacatggtcatcaccgtgaagcggcaccctcagaaagtgcacaaccaggacagcgaggacgagctgtaggcgccgtcgac

SEQ ID NO: 32 - DNA, 3428 bp, synthetic construct
  misc_feature 1..3428: note = Construct comprising EFS promoter and COSEQ2-BBS10 nucleotide sequence (referred to as EFS-COSEQ2-BBS10)
  source 1..3428: mol_type = other DNA
actagtgggcagatctcgatcgagttgggccccagagcttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccatgagcttcgaattcgccaccatgctgagctccatggctgccgccggatctgtgaaagccgccctgcaggtggcagaggtgctggaggcaatcgtgagctgttgcgtgggacctgagggccggcaggtgctgtgcacaaagccaaccggcgaggtgctgctgtctagaaatggcggccggctgctggaggccctgcacctggagcacccaattgcaagaatgatcgtggactgcgtgtctagccacctgaagaagacaggcgatggcgccaagaccttcatcatcttcctgtgccacctgctgaggggcctgcacgccatcacagaccgcgagaaggatcctctgatgtgcgagaacatccagacccacggcaggcactggaagaattgttcccgctggaagttcatctctcaggccctgctgacatttcagacccagatcctggacggcatcatggatcagtatctgagcaggcacttcctgtccatcttttcctctgccaaggagcggaccctgtgcagaagctccctggagctgctgctggaggcctacttctgtggccgggtgggcagaaacaatcacaagtttatcagccagctgatgtgcgactatttctttaagtgcatgacctgtaagtccggcatcggcgtgttcgagctggtggacgatcactttgtggagctgaacgtgggagtgacaggcctgcccgtgtccgactctcgcatcatcgccggcctggtgctgcagaaggatttctccgtgtaccggcctgccgacggcgatatgagaatggtcatcgtgaccgagacaatccagccactgttcagcacctccggctctgagttcatcctgaacagcgaggcccagttccagacatctcagttttggatcatggagaagaccaaggccatcatgaagcacctgcacagccagaacgtgaagctgctgatctctagcgtgaagcagccagacctggtgtcttactatgccggcgtgaatggcatcagcgtggtggagtgtctgtcctctgaggaggtgtccctgatccggagaatcattggcctgtctcccttcgtgccccctcaggcctttagccagtgcgagatccccaacacagccctggtgaagttctgtaagcctctgatcctgaggtccaagcggtacgtgcacctgggcctgatcagcacctgcgcctttatcccacactctatcgtgctgtgcggacctgtgcacggcctgattgagcagcacgaggatgcactgcacggcgccctgaagatgctgaggcagctgttcaaggacctggatctgaattacatgacccagacaaacgaccagaatggcacaagctccctgtttatctacaagaactctggcgagagctatcaggccccagatcccggcaatggcagcattcagcgcccctaccaggacacagtggcagagaacaaggatgccctggagaagacccagacatatctgaaggtgcactccaacctggtcatccctgacgtggagctggagacatacatcccttattctaccccaacactgacccccacagataccttccagacagtggagacactgacctgcctgtccctggagaggaaccgcctgaccgactactatgagcccctgctgaagaacaattccacagcctactctacccggggcaatagaatcgagatcagctatgagaacctgcaggtgacaaatatcaccagaaagggctctatgctgcctgtgagctgcaagctgccaaacatgggcaccagccagtcctacctgtctagctccatgcctgcaggatgcgtgctgcctgtgggcggcaacttcgagatcctgctgcactactatctgctgaattatgccaagaagtgccaccagagcgaggagacaatggtgtccatgatcatcgccaacgccctgctgggcatcccaaaggtgctgtacaagtctaagaccggcaagtacagctttccccacacctatatccgggccgtgcacgccctgcagacaaatcagcctctggtgtctagccagaccggcctggagtccgtgatgggcaagtaccagctgctgacatctgtgctgcagtgtctgacaaagatcctgaccatcgatatggtcatcaccgtgaagagacacccacagaaggtgcacaatcaggacagcgaggatgagctgtaagcggccgcgtcgac

SEQ ID NO: 33 - DNA, 3470 bp, synthetic construct
  misc_feature 1..3470: note = Construct comprising UBC promoter and wild type BBS10 nucleotide sequence (referred to as UBC-WTBBS10)
  source 1..3470: mol_type = other DNA
actagtaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgagctcaagcttcgaattcgccaccatgttaagttctatggccgctgcagggtctgtgaaggcggcgttgcaggtggccgaggtgctggaagccatcgtgagctgctgcgtggggcccgagggacggcaagttttgtgtacgaagcccactggcgaggtgcttctcagccggaatggaggccgcctcctggaggcgctacacttagagcatcccatagccaggatgatagtggactgtgtttccagtcatctcaaaaaaacaggagatggtgcaaaaacatttattatctttctttgccatttgcttagaggacttcatgcaatcacagacagagaaaaggatcctttgatgtgtgaaaacattcaaacccatggaaggcattggaaaaattgttctcggtggaaatttatttcccaggctctcctaacgtttcagacacaaatattagacggtattatggaccagtacctaagtagacactttttgtctatcttttcgtctgctaaagagagaacattgtgtaggagctctttagagttgctcttagaagcatacttttgtggaagagtgggaagaaataatcataaatttatttcacagttgatgtgtgactactttttcaagtgtatgacttgtaaaagtgggattggtgtatttgagttagtggatgaccattttgtagagttgaatgttggtgtcactggccttcctgtttcagattccaggatcatagctggtcttgtgcttcagaaagatttttctgtgtaccgcccagcagatggtgacatgcgaatggtgatagtaacagaaaccattcagcctcttttttccacttctggatcagagtttattctaaattcagaagcacagtttcagacatctcaattttggattatggaaaagacaaaagcaataatgaaacatctacatagtcagaatgtaaaattgctcatatctagtgtgaaacaaccagatttagttagttattatgcaggggtgaatggcatatcagtggttgagtgtttatcatcagaagaagtttctcttatccggaggatcattggtctttctccatttgtaccaccacaggccttttcgcagtgtgaaatacctaacactgctttggtgaaattttgtaaacctcttatccttagatccaaaagatatgttcatctaggcttgataagcacatgtgcatttataccacactctatagttctttgtggaccagtgcatggtctcattgaacaacatgaggatgctttacatggagcacttaaaatgcttcggcaattatttaaagaccttgatctaaattacatgacacaaaccaatgaccaaaatggcacttcaagtctttttatttataagaacagtggagaaagttatcaagcaccagatcctggtaatggctcaatacaaaggccttatcaggacacagttgcagagaacaaagatgcattggaaaaaactcaaacatatttaaaagtacattctaatttggtaattccagatgtagaattagaaacatatattccgtattcaacccccacactgacaccaacagatacattccaaacagttgaaacgctgacatgtttgtctttggaaagaaacaggctaactgattattatgaaccattactcaagaacaattccactgcttattcaacaaggggaaatagaatagaaatttcttacgaaaatttacaggtcacaaatattactagaaagggaagcatgttaccagtgagctgtaagttaccgaatatgggtacttcccagagttacctttcctcatctatgccagctggttgtgttttgccagtaggtggtaattttgagatcttgttacattactatcttctcaattatgccaaaaaatgccatcaatcagaagaaaccatggttagtatgataatagctaatgcacttttaggcattcccaaagtcctttataaatctaaaacaggaaagtacagctttccacatacatatataagagctgtccatgcactgcaaaccaatcaacccttggtaagcagtcagacaggtttggaatcagtaatgggtaaataccagctactaacttcagttcttcagtgtttgacaaaaatattaaccattgacatggtaatcactgttaagagacaccctcagaaagttcacaatcaagattcagaagatgaactataagcggccgcgtcgac

SEQ ID NO: 34 - DNA, 3467 bp, synthetic construct
  misc_feature 1..3467: note = Construct comprising UBC promoter and COSEQ1-BBS10 nucleotide sequence (referred to as UBC-COSEQ1BBS10)
  source 1..3467: mol_type = other DNA
actagtaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgagctcaagcttcgaattcgccaccatgctgtctagcatggctgccgctggatctgtgaaagctgctctgcaggttgccgaggtgctggaagccatcgtgtcttgttgtgtgggacctgagggcagacaggtgctgtgtacaaagcctacaggcgaagtgctgctgagcagaaatggcggcagactgctcgaagccctgcacctggaacaccctatcgccagaatgatcgtggactgcgtgtccagccacctgaagaaaacaggcgacggcgccaagaccttcatcatctttctgtgccatctgctgcggggcctgcacgccatcaccgatagagaaaaggaccctctgatgtgcgagaacatccagacacacggccggcactggaagaactgctcccggtggaagtttatcagccaggctctgctgacctttcagacccagatcctggacggcatcatggaccagtacctgagcagacacttcctgagcatcttcagcagcgccaaagaacggaccctgtgcagaagctctctggaactgctgctcgaggcctacttttgcggcagagtgggcagaaacaaccacaagttcatctcccagctgatgtgtgactacttcttcaagtgcatgacctgcaagagcggcatcggcgtgttcgagctggtggacgatcacttcgtggaactgaatgtgggcgtgaccggcctgcctgtgtccgatagcagaattattgccggcctggtgctgcagaaagacttcagcgtgtacagacccgccgacggcgacatgagaatggtcatcgtgaccgagacaatccagcctctgttcagcacaagcggcagcgagttcatcctgaacagcgaggcccagtttcagaccagccagttctggatcatggaaaagaccaaggccatcatgaagcacctccacagccagaacgtgaagctgctgatctccagcgtgaagcagcccgacctggtgtcttattatgccggcgtgaacggcatcagcgtggtggaatgtctgagcagcgaagaggtgtccctgatcagacggatcatcggactgagcccctttgtgcctcctcaagcctttagccagtgcgagatccctaacacagccctggtcaagttctgcaagcccctgatcctgcggagcaagagatatgtgcacctgggcctgatcagcacatgcgccttcattcctcactccatcgtgctgtgtggacctgtgcacggactgattgagcagcacgaagatgcactgcacggcgccctgaaaatgctgagacagctgttcaaggacctggacctgaactacatgacccagaccaacgaccagaacggcaccagcagcctgttcatctacaagaacagcggcgagagctatcaggccccagatccaggcaatggcagcatccagaggccttaccaggataccgtggccgagaacaaggacgccctggaaaaaacccagacctacctgaaggtgcacagcaacctggtcatccccgatgtggaactggaaacctacattccctacagcacccctacactgacccctaccgataccttccagaccgtggaaaccctgacctgtctgagcctggaacggaacagactgaccgactactacgagcccctgctgaaaaacaacagcaccgcctatagcacccggggcaacagaatcgagatcagctacgagaacctgcaagtgaccaacatcacccggaagggctccatgctgccagtgtcctgcaagctgcctaatatgggcaccagccagagctacctgtcctcttctatgcctgccggatgtgtgctgcctgtcggcggcaattttgagatcctgctgcactactacctgctgaactacgccaagaagtgccaccagagcgaagagacaatggtgtccatgattatcgccaacgctctgctgggcatccccaaggtgctgtacaagagcaagaccggcaagtacagcttccctcacacctacattagagccgtgcacgccctgcagaccaatcagccactggtttctagccagacaggcctggaaagcgtgatgggaaagtaccagctgctgaccagcgtgctgcagtgcctgaccaagatcctgaccatcgacatggtcatcaccgtgaagcggcaccctcagaaagtgcacaaccaggacagcgaggacgagctgtaggcgccgtcgac

SEQ ID NO: 35 - DNA, 3470 bp, synthetic construct
  misc_feature 1..3470: note = Construct comprising UBC promoter and COSEQ2-BBS10 nucleotide sequence (referred to as UBC-COSEQ2-BBS10)
  source 1..3470: mol_type = other DNA
actagtaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgagctcaagcttcgaattcgccaccatgctgagctccatggctgccgccggatctgtgaaagccgccctgcaggtggcagaggtgctggaggcaatcgtgagctgttgcgtgggacctgagggccggcaggtgctgtgcacaaagccaaccggcgaggtgctgctgtctagaaatggcggccggctgctggaggccctgcacctggagcacccaattgcaagaatgatcgtggactgcgtgtctagccacctgaagaagacaggcgatggcgccaagaccttcatcatcttcctgtgccacctgctgaggggcctgcacgccatcacagaccgcgagaaggatcctctgatgtgcgagaacatccagacccacggcaggcactggaagaattgttcccgctggaagttcatctctcaggccctgctgacatttcagacccagatcctggacggcatcatggatcagtatctgagcaggcacttcctgtccatcttttcctctgccaaggagcggaccctgtgcagaagctccctggagctgctgctggaggcctacttctgtggccgggtgggcagaaacaatcacaagtttatcagccagctgatgtgcgactatttctttaagtgcatgacctgtaagtccggcatcggcgtgttcgagctggtggacgatcactttgtggagctgaacgtgggagtgacaggcctgcccgtgtccgactctcgcatcatcgccggcctggtgctgcagaaggatttctccgtgtaccggcctgccgacggcgatatgagaatggtcatcgtgaccgagacaatccagccactgttcagcacctccggctctgagttcatcctgaacagcgaggcccagttccagacatctcagttttggatcatggagaagaccaaggccatcatgaagcacctgcacagccagaacgtgaagctgctgatctctagcgtgaagcagccagacctggtgtcttactatgccggcgtgaatggcatcagcgtggtggagtgtctgtcctctgaggaggtgtccctgatccggagaatcattggcctgtctcccttcgtgccccctcaggcctttagccagtgcgagatccccaacacagccctggtgaagttctgtaagcctctgatcctgaggtccaagcggtacgtgcacctgggcctgatcagcacctgcgcctttatcccacactctatcgtgctgtgcggacctgtgcacggcctgattgagcagcacgaggatgcactgcacggcgccctgaagatgctgaggcagctgttcaaggacctggatctgaattacatgacccagacaaacgaccagaatggcacaagctccctgtttatctacaagaactctggcgagagctatcaggccccagatcccggcaatggcagcattcagcgcccctaccaggacacagtggcagagaacaaggatgccctggagaagacccagacatatctgaaggtgcactccaacctggtcatccctgacgtggagctggagacatacatcccttattctaccccaacactgacccccacagataccttccagacagtggagacactgacctgcctgtccctggagaggaaccgcctgaccgactactatgagcccctgctgaagaacaattccacagcctactctacccggggcaatagaatcgagatcagctatgagaacctgcaggtgacaaatatcaccagaaagggctctatgctgcctgtgagctgcaagctgccaaacatgggcaccagccagtcctacctgtctagctccatgcctgcaggatgcgtgctgcctgtgggcggcaacttcgagatcctgctgcactactatctgctgaattatgccaagaagtgccaccagagcgaggagacaatggtgtccatgatcatcgccaacgccctgctgggcatcccaaaggtgctgtacaagtctaagaccggcaagtacagctttccccacacctatatccgggccgtgcacgccctgcagacaaatcagcctctggtgtctagccagaccggcctggagtccgtgatgggcaagtaccagctgctgacatctgtgctgcagtgtctgacaaagatcctgaccatcgatatggtcatcaccgtgaagagacacccacagaaggtgcacaatcaggacagcgaggatgagctgtaagcggccgcgtcgac

SEQ ID NO: 36 - DNA, 2815 bp, synthetic construct
  misc_feature 1..2815: note = Construct comprising CMV promoter and wild type BBS10 nucleotide sequence (referred to as CMV-WTBBS10)
  source 1..2815: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatgttaagttctatggccgctgcagggtctgtgaaggcggcgttgcaggtggccgaggtgctggaagccatcgtgagctgctgcgtggggcccgagggacggcaagttttgtgtacgaagcccactggcgaggtgcttctcagccggaatggaggccgcctcctggaggcgctacacttagagcatcccatagccaggatgatagtggactgtgtttccagtcatctcaaaaaaacaggagatggtgcaaaaacatttattatctttctttgccatttgcttagaggacttcatgcaatcacagacagagaaaaggatcctttgatgtgtgaaaacattcaaacccatggaaggcattggaaaaattgttctcggtggaaatttatttcccaggctctcctaacgtttcagacacaaatattagacggtattatggaccagtacctaagtagacactttttgtctatcttttcgtctgctaaagagagaacattgtgtaggagctctttagagttgctcttagaagcatacttttgtggaagagtgggaagaaataatcataaatttatttcacagttgatgtgtgactactttttcaagtgtatgacttgtaaaagtgggattggtgtatttgagttagtggatgaccattttgtagagttgaatgttggtgtcactggccttcctgtttcagattccaggatcatagctggtcttgtgcttcagaaagatttttctgtgtaccgcccagcagatggtgacatgcgaatggtgatagtaacagaaaccattcagcctcttttttccacttctggatcagagtttattctaaattcagaagcacagtttcagacatctcaattttggattatggaaaagacaaaagcaataatgaaacatctacatagtcagaatgtaaaattgctcatatctagtgtgaaacaaccagatttagttagttattatgcaggggtgaatggcatatcagtggttgagtgtttatcatcagaagaagtttctcttatccggaggatcattggtctttctccatttgtaccaccacaggccttttcgcagtgtgaaatacctaacactgctttggtgaaattttgtaaacctcttatccttagatccaaaagatatgttcatctaggcttgataagcacatgtgcatttataccacactctatagttctttgtggaccagtgcatggtctcattgaacaacatgaggatgctttacatggagcacttaaaatgcttcggcaattatttaaagaccttgatctaaattacatgacacaaaccaatgaccaaaatggcacttcaagtctttttatttataagaacagtggagaaagttatcaagcaccagatcctggtaatggctcaatacaaaggccttatcaggacacagttgcagagaacaaagatgcattggaaaaaactcaaacatatttaaaagtacattctaatttggtaattccagatgtagaattagaaacatatattccgtattcaacccccacactgacaccaacagatacattccaaacagttgaaacgctgacatgtttgtctttggaaagaaacaggctaactgattattatgaaccattactcaagaacaattccactgcttattcaacaaggggaaatagaatagaaatttcttacgaaaatttacaggtcacaaatattactagaaagggaagcatgttaccagtgagctgtaagttaccgaatatgggtacttcccagagttacctttcctcatctatgccagctggttgtgttttgccagtaggtggtaattttgagatcttgttacattactatcttctcaattatgccaaaaaatgccatcaatcagaagaaaccatggttagtatgataatagctaatgcacttttaggcattcccaaagtcctttataaatctaaaacaggaaagtacagctttccacatacatatataagagctgtccatgcactgcaaaccaatcaacccttggtaagcagtcagacaggtttggaatcagtaatgggtaaataccagctactaacttcagttcttcagtgtttgacaaaaatattaaccattgacatggtaatcactgttaagagacaccctcagaaagttcacaatcaagattcagaagatgaactataagcggccgcgtcgac

SEQ ID NO: 37 - DNA, 2812 bp, synthetic construct
  misc_feature 1..2812: note = Construct comprising CMV promoter and COSEQ1-BBS10 nucleotide sequence (referred to as CMV-COSEQ1-BBS10)
  source 1..2812: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatgctgtctagcatggctgccgctggatctgtgaaagctgctctgcaggttgccgaggtgctggaagccatcgtgtcttgttgtgtgggacctgagggcagacaggtgctgtgtacaaagcctacaggcgaagtgctgctgagcagaaatggcggcagactgctcgaagccctgcacctggaacaccctatcgccagaatgatcgtggactgcgtgtccagccacctgaagaaaacaggcgacggcgccaagaccttcatcatctttctgtgccatctgctgcggggcctgcacgccatcaccgatagagaaaaggaccctctgatgtgcgagaacatccagacacacggccggcactggaagaactgctcccggtggaagtttatcagccaggctctgctgacctttcagacccagatcctggacggcatcatggaccagtacctgagcagacacttcctgagcatcttcagcagcgccaaagaacggaccctgtgcagaagctctctggaactgctgctcgaggcctacttttgcggcagagtgggcagaaacaaccacaagttcatctcccagctgatgtgtgactacttcttcaagtgcatgacctgcaagagcggcatcggcgtgttcgagctggtggacgatcacttcgtggaactgaatgtgggcgtgaccggcctgcctgtgtccgatagcagaattattgccggcctggtgctgcagaaagacttcagcgtgtacagacccgccgacggcgacatgagaatggtcatcgtgaccgagacaatccagcctctgttcagcacaagcggcagcgagttcatcctgaacagcgaggcccagtttcagaccagccagttctggatcatggaaaagaccaaggccatcatgaagcacctccacagccagaacgtgaagctgctgatctccagcgtgaagcagcccgacctggtgtcttattatgccggcgtgaacggcatcagcgtggtggaatgtctgagcagcgaagaggtgtccctgatcagacggatcatcggactgagcccctttgtgcctcctcaagcctttagccagtgcgagatccctaacacagccctggtcaagttctgcaagcccctgatcctgcggagcaagagatatgtgcacctgggcctgatcagcacatgcgccttcattcctcactccatcgtgctgtgtggacctgtgcacggactgattgagcagcacgaagatgcactgcacggcgccctgaaaatgctgagacagctgttcaaggacctggacctgaactacatgacccagaccaacgaccagaacggcaccagcagcctgttcatctacaagaacagcggcgagagctatcaggccccagatccaggcaatggcagcatccagaggccttaccaggataccgtggccgagaacaaggacgccctggaaaaaacccagacctacctgaaggtgcacagcaacctggtcatccccgatgtggaactggaaacctacattccctacagcacccctacactgacccctaccgataccttccagaccgtggaaaccctgacctgtctgagcctggaacggaacagactgaccgactactacgagcccctgctgaaaaacaacagcaccgcctatagcacccggggcaacagaatcgagatcagctacgagaacctgcaagtgaccaacatcacccggaagggctccatgctgccagtgtcctgcaagctgcctaatatgggcaccagccagagctacctgtcctcttctatgcctgccggatgtgtgctgcctgtcggcggcaattttgagatcctgctgcactactacctgctgaactacgccaagaagtgccaccagagcgaagagacaatggtgtccatgattatcgccaacgctctgctgggcatccccaaggtgctgtacaagagcaagaccggcaagtacagcttccctcacacctacattagagccgtgcacgccctgcagaccaatcagccactggtttctagccagacaggcctggaaagcgtgatgggaaagtaccagctgctgaccagcgtgctgcagtgcctgaccaagatcctgaccatcgacatggtcatcaccgtgaagcggcaccctcagaaagtgcacaaccaggacagcgaggacgagctgtaggcgccgtcgac

SEQ ID NO: 38 - DNA, 2815 bp, synthetic construct
  misc_feature 1..2815: note = Construct comprising CMV promoter and COSEQ2-BBS10 nucleotide sequence (referred to as CMV-COSEQ2-BBS10)
  source 1..2815: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatgctgagctccatggctgccgccggatctgtgaaagccgccctgcaggtggcagaggtgctggaggcaatcgtgagctgttgcgtgggacctgagggccggcaggtgctgtgcacaaagccaaccggcgaggtgctgctgtctagaaatggcggccggctgctggaggccctgcacctggagcacccaattgcaagaatgatcgtggactgcgtgtctagccacctgaagaagacaggcgatggcgccaagaccttcatcatcttcctgtgccacctgctgaggggcctgcacgccatcacagaccgcgagaaggatcctctgatgtgcgagaacatccagacccacggcaggcactggaagaattgttcccgctggaagttcatctctcaggccctgctgacatttcagacccagatcctggacggcatcatggatcagtatctgagcaggcacttcctgtccatcttttcctctgccaaggagcggaccctgtgcagaagctccctggagctgctgctggaggcctacttctgtggccgggtgggcagaaacaatcacaagtttatcagccagctgatgtgcgactatttctttaagtgcatgacctgtaagtccggcatcggcgtgttcgagctggtggacgatcactttgtggagctgaacgtgggagtgacaggcctgcccgtgtccgactctcgcatcatcgccggcctggtgctgcagaaggatttctccgtgtaccggcctgccgacggcgatatgagaatggtcatcgtgaccgagacaatccagccactgttcagcacctccggctctgagttcatcctgaacagcgaggcccagttccagacatctcagttttggatcatggagaagaccaaggccatcatgaagcacctgcacagccagaacgtgaagctgctgatctctagcgtgaagcagccagacctggtgtcttactatgccggcgtgaatggcatcagcgtggtggagtgtctgtcctctgaggaggtgtccctgatccggagaatcattggcctgtctcccttcgtgccccctcaggcctttagccagtgcgagatccccaacacagccctggtgaagttctgtaagcctctgatcctgaggtccaagcggtacgtgcacctgggcctgatcagcacctgcgcctttatcccacactctatcgtgctgtgcggacctgtgcacggcctgattgagcagcacgaggatgcactgcacggcgccctgaagatgctgaggcagctgttcaaggacctggatctgaattacatgacccagacaaacgaccagaatggcacaagctccctgtttatctacaagaactctggcgagagctatcaggccccagatcccggcaatggcagcattcagcgcccctaccaggacacagtggcagagaacaaggatgccctggagaagacccagacatatctgaaggtgcactccaacctggtcatccctgacgtggagctggagacatacatcccttattctaccccaacactgacccccacagataccttccagacagtggagacactgacctgcctgtccctggagaggaaccgcctgaccgactactatgagcccctgctgaagaacaattccacagcctactctacccggggcaatagaatcgagatcagctatgagaacctgcaggtgacaaatatcaccagaaagggctctatgctgcctgtgagctgcaagctgccaaacatgggcaccagccagtcctacctgtctagctccatgcctgcaggatgcgtgctgcctgtgggcggcaacttcgagatcctgctgcactactatctgctgaattatgccaagaagtgccaccagagcgaggagacaatggtgtccatgatcatcgccaacgccctgctgggcatcccaaaggtgctgtacaagtctaagaccggcaagtacagctttccccacacctatatccgggccgtgcacgccctgcagacaaatcagcctctggtgtctagccagaccggcctggagtccgtgatgggcaagtaccagctgctgacatctgtgctgcagtgtctgacaaagatcctgaccatcgatatggtcatcaccgtgaagagacacccacagaaggtgcacaatcaggacagcgaggatgagctgtaagcggccgcgtcgac

SEQ ID NO: 39 - DNA, 2658 bp, synthetic construct
  misc_feature 1..2658: note = Construct comprising CBA promoter and wild type BBS10 nucleotide sequence (referred to as CBA-WTBBS10)
  source 1..2658: mol_type = other DNA
actagtgtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatgttaagttctatggccgctgcagggtctgtgaaggcggcgttgcaggtggccgaggtgctggaagccatcgtgagctgctgcgtggggcccgagggacggcaagttttgtgtacgaagcccactggcgaggtgcttctcagccggaatggaggccgcctcctggaggcgctacacttagagcatcccatagccaggatgatagtggactgtgtttccagtcatctcaaaaaaacaggagatggtgcaaaaacatttattatctttctttgccatttgcttagaggacttcatgcaatcacagacagagaaaaggatcctttgatgtgtgaaaacattcaaacccatggaaggcattggaaaaattgttctcggtggaaatttatttcccaggctctcctaacgtttcagacacaaatattagacggtattatggaccagtacctaagtagacactttttgtctatcttttcgtctgctaaagagagaacattgtgtaggagctctttagagttgctcttagaagcatacttttgtggaagagtgggaagaaataatcataaatttatttcacagttgatgtgtgactactttttcaagtgtatgacttgtaaaagtgggattggtgtatttgagttagtggatgaccattttgtagagttgaatgttggtgtcactggccttcctgtttcagattccaggatcatagctggtcttgtgcttcagaaagatttttctgtgtaccgcccagcagatggtgacatgcgaatggtgatagtaacagaaaccattcagcctcttttttccacttctggatcagagtttattctaaattcagaagcacagtttcagacatctcaattttggattatggaaaagacaaaagcaataatgaaacatctacatagtcagaatgtaaaattgctcatatctagtgtgaaacaaccagatttagttagttattatgcaggggtgaatggcatatcagtggttgagtgtttatcatcagaagaagtttctcttatccggaggatcattggtctttctccatttgtaccaccacaggccttttcgcagtgtgaaatacctaacactgctttggtgaaattttgtaaacctcttatccttagatccaaaagatatgttcatctaggcttgataagcacatgtgcatttataccacactctatagttctttgtggaccagtgcatggtctcattgaacaacatgaggatgctttacatggagcacttaaaatgcttcggcaattatttaaagaccttgatctaaattacatgacacaaaccaatgaccaaaatggcacttcaagtctttttatttataagaacagtggagaaagttatcaagcaccagatcctggtaatggctcaatacaaaggccttatcaggacacagttgcagagaacaaagatgcattggaaaaaactcaaacatatttaaaagtacattctaatttggtaattccagatgtagaattagaaacatatattccgtattcaacccccacactgacaccaacagatacattccaaacagttgaaacgctgacatgtttgtctttggaaagaaacaggctaactgattattatgaaccattactcaagaacaattccactgcttattcaacaaggggaaatagaatagaaatttcttacgaaaatttacaggtcacaaatattactagaaagggaagcatgttaccagtgagctgtaagttaccgaatatgggtacttcccagagttacctttcctcatctatgccagctggttgtgttttgccagtaggtggtaattttgagatcttgttacattactatcttctcaattatgccaaaaaatgccatcaatcagaagaaaccatggttagtatgataatagctaatgcacttttaggcattcccaaagtcctttataaatctaaaacaggaaagtacagctttccacatacatatataagagctgtccatgcactgcaaaccaatcaacccttggtaagcagtcagacaggtttggaatcagtaatgggtaaataccagctactaacttcagttcttcagtgtttgacaaaaatattaaccattgacatggtaatcactgttaagagacaccctcagaaagttcacaatcaagattcagaagatgaactataagcggccgcgtcgac

SEQ ID NO: 40 - DNA, 2655 bp, synthetic construct
  misc_feature 1..2655: note = Construct comprising CBA promoter and COSEQ1-BBS10 nucleotide sequence (referred to as CBA-COSEQ1-BBS10)
  source 1..2655: mol_type = other DNA
actagtgtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatgctgtctagcatggctgccgctggatctgtgaaagctgctctgcaggttgccgaggtgctggaagccatcgtgtcttgttgtgtgggacctgagggcagacaggtgctgtgtacaaagcctacaggcgaagtgctgctgagcagaaatggcggcagactgctcgaagccctgcacctggaacaccctatcgccagaatgatcgtggactgcgtgtccagccacctgaagaaaacaggcgacggcgccaagaccttcatcatctttctgtgccatctgctgcggggcctgcacgccatcaccgatagagaaaaggaccctctgatgtgcgagaacatccagacacacggccggcactggaagaactgctcccggtggaagtttatcagccaggctctgctgacctttcagacccagatcctggacggcatcatggaccagtacctgagcagacacttcctgagcatcttcagcagcgccaaagaacggaccctgtgcagaagctctctggaactgctgctcgaggcctacttttgcggcagagtgggcagaaacaaccacaagttcatctcccagctgatgtgtgactacttcttcaagtgcatgacctgcaagagcggcatcggcgtgttcgagctggtggacgatcacttcgtggaactgaatgtgggcgtgaccggcctgcctgtgtccgatagcagaattattgccggcctggtgctgcagaaagacttcagcgtgtacagacccgccgacggcgacatgagaatggtcatcgtgaccgagacaatccagcctctgttcagcacaagcggcagcgagttcatcctgaacagcgaggcccagtttcagaccagccagttctggatcatggaaaagaccaaggccatcatgaagcacctccacagccagaacgtgaagctgctgatctccagcgtgaagcagcccgacctggtgtcttattatgccggcgtgaacggcatcagcgtggtggaatgtctgagcagcgaagaggtgtccctgatcagacggatcatcggactgagcccctttgtgcctcctcaagcctttagccagtgcgagatccctaacacagccctggtcaagttctgcaagcccctgatcctgcggagcaagagatatgtgcacctgggcctgatcagcacatgcgccttcattcctcactccatcgtgctgtgtggacctgtgcacggactgattgagcagcacgaagatgcactgcacggcgccctgaaaatgctgagacagctgttcaaggacctggacctgaactacatgacccagaccaacgaccagaacggcaccagcagcctgttcatctacaagaacagcggcgagagctatcaggccccagatccaggcaatggcagcatccagaggccttaccaggataccgtggccgagaacaaggacgccctggaaaaaacccagacctacctgaaggtgcacagcaacctggtcatccccgatgtggaactggaaacctacattccctacagcacccctacactgacccctaccgataccttccagaccgtggaaaccctgacctgtctgagcctggaacggaacagactgaccgactactacgagcccctgctgaaaaacaacagcaccgcctatagcacccggggcaacagaatcgagatcagctacgagaacctgcaagtgaccaacatcacccggaagggctccatgctgccagtgtcctgcaagctgcctaatatgggcaccagccagagctacctgtcctcttctatgcctgccggatgtgtgctgcctgtcggcggcaattttgagatcctgctgcactactacctgctgaactacgccaagaagtgccaccagagcgaagagacaatggtgtccatgattatcgccaacgctctgctgggcatccccaaggtgctgtacaagagcaagaccggcaagtacagcttccctcacacctacattagagccgtgcacgccctgcagaccaatcagccactggtttctagccagacaggcctggaaagcgtgatgggaaagtaccagctgctgaccagcgtgctgcagtgcctgaccaagatcctgaccatcgacatggtcatcaccgtgaagcggcaccctcagaaagtgcacaaccaggacagcgaggacgagctgtaggcgccgtcgac

SEQ ID NO: 41 - DNA, 2658 bp, synthetic construct
  misc_feature 1..2658: note = Construct comprising CBA promoter and COSEQ2-BBS10 nucleotide sequence (referred to as CBA-COSEQ2-BBS10)
  source 1..2658: mol_type = other DNA
actagtgtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatgctgagctccatggctgccgccggatctgtgaaagccgccctgcaggtggcagaggtgctggaggcaatcgtgagctgttgcgtgggacctgagggccggcaggtgctgtgcacaaagccaaccggcgaggtgctgctgtctagaaatggcggccggctgctggaggccctgcacctggagcacccaattgcaagaatgatcgtggactgcgtgtctagccacctgaagaagacaggcgatggcgccaagaccttcatcatcttcctgtgccacctgctgaggggcctgcacgccatcacagaccgcgagaaggatcctctgatgtgcgagaacatccagacccacggcaggcactggaagaattgttcccgctggaagttcatctctcaggccctgctgacatttcagacccagatcctggacggcatcatggatcagtatctgagcaggcacttcctgtccatcttttcctctgccaaggagcggaccctgtgcagaagctccctggagctgctgctggaggcctacttctgtggccgggtgggcagaaacaatcacaagtttatcagccagctgatgtgcgactatttctttaagtgcatgacctgtaagtccggcatcggcgtgttcgagctggtggacgatcactttgtggagctgaacgtgggagtgacaggcctgcccgtgtccgactctcgcatcatcgccggcctggtgctgcagaaggatttctccgtgtaccggcctgccgacggcgatatgagaatggtcatcgtgaccgagacaatccagccactgttcagcacctccggctctgagttcatcctgaacagcgaggcccagttccagacatctcagttttggatcatggagaagaccaaggccatcatgaagcacctgcacagccagaacgtgaagctgctgatctctagcgtgaagcagccagacctggtgtcttactatgccggcgtgaatggcatcagcgtggtggagtgtctgtcctctgaggaggtgtccctgatccggagaatcattggcctgtctcccttcgtgccccctcaggcctttagccagtgcgagatccccaacacagccctggtgaagttctgtaagcctctgatcctgaggtccaagcggtacgtgcacctgggcctgatcagcacctgcgcctttatcccacactctatcgtgctgtgcggacctgtgcacggcctgattgagcagcacgaggatgcactgcacggcgccctgaagatgctgaggcagctgttcaaggacctggatctgaattacatgacccagacaaacgaccagaatggcacaagctccctgtttatctacaagaactctggcgagagctatcaggccccagatcccggcaatggcagcattcagcgcccctaccaggacacagtggcagagaacaaggatgccctggagaagacccagacatatctgaaggtgcactccaacctggtcatccctgacgtggagctggagacatacatcccttattctaccccaacactgacccccacagataccttccagacagtggagacactgacctgcctgtccctggagaggaaccgcctgaccgactactatgagcccctgctgaagaacaattccacagcctactctacccggggcaatagaatcgagatcagctatgagaacctgcaggtgacaaatatcaccagaaagggctctatgctgcctgtgagctgcaagctgccaaacatgggcaccagccagtcctacctgtctagctccatgcctgcaggatgcgtgctgcctgtgggcggcaacttcgagatcctgctgcactactatctgctgaattatgccaagaagtgccaccagagcgaggagacaatggtgtccatgatcatcgccaacgccctgctgggcatcccaaaggtgctgtacaagtctaagaccggcaagtacagctttccccacacctatatccgggccgtgcacgccctgcagacaaatcagcctctggtgtctagccagaccggcctggagtccgtgatgggcaagtaccagctgctgacatctgtgctgcagtgtctgacaaagatcctgaccatcgatatggtcatcaccgtgaagagacacccacagaaggtgcacaatcaggacagcgaggatgagctgtaagcggccgcgtcgac

SEQ ID NO: 42 - DNA, 2901 bp, synthetic construct
  misc_feature 1..2901: note = Construct comprising CAG promoter and wild type BBS10 nucleotide sequence (referred to as CAG-WTBBS10)
  source 1..2901: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatgttaagttctatggccgctgcagggtctgtgaaggcggcgttgcaggtggccgaggtgctggaagccatcgtgagctgctgcgtggggcccgagggacggcaagttttgtgtacgaagcccactggcgaggtgcttctcagccggaatggaggccgcctcctggaggcgctacacttagagcatcccatagccaggatgatagtggactgtgtttccagtcatctcaaaaaaacaggagatggtgcaaaaacatttattatctttctttgccatttgcttagaggacttcatgcaatcacagacagagaaaaggatcctttgatgtgtgaaaacattcaaacccatggaaggcattggaaaaattgttctcggtggaaatttatttcccaggctctcctaacgtttcagacacaaatattagacggtattatggaccagtacctaagtagacactttttgtctatcttttcgtctgctaaagagagaacattgtgtaggagctctttagagttgctcttagaagcatacttttgtggaagagtgggaagaaataatcataaatttatttcacagttgatgtgtgactactttttcaagtgtatgacttgtaaaagtgggattggtgtatttgagttagtggatgaccattttgtagagttgaatgttggtgtcactggccttcctgtttcagattccaggatcatagctggtcttgtgcttcagaaagatttttctgtgtaccgcccagcagatggtgacatgcgaatggtgatagtaacagaaaccattcagcctcttttttccacttctggatcagagtttattctaaattcagaagcacagtttcagacatctcaattttggattatggaaaagacaaaagcaataatgaaacatctacatagtcagaatgtaaaattgctcatatctagtgtgaaacaaccagatttagttagttattatgcaggggtgaatggcatatcagtggttgagtgtttatcatcagaagaagtttctcttatccggaggatcattggtctttctccatttgtaccaccacaggccttttcgcagtgtgaaatacctaacactgctttggtgaaattttgtaaacctcttatccttagatccaaaagatatgttcatctaggcttgataagcacatgtgcatttataccacactctatagttctttgtggaccagtgcatggtctcattgaacaacatgaggatgctttacatggagcacttaaaatgcttcggcaattatttaaagaccttgatctaaattacatgacacaaaccaatgaccaaaatggcacttcaagtctttttatttataagaacagtggagaaagttatcaagcaccagatcctggtaatggctcaatacaaaggccttatcaggacacagttgcagagaacaaagatgcattggaaaaaactcaaacatatttaaaagtacattctaatttggtaattccagatgtagaattagaaacatatattccgtattcaacccccacactgacaccaacagatacattccaaacagttgaaacgctgacatgtttgtctttggaaagaaacaggctaactgattattatgaaccattactcaagaacaattccactgcttattcaacaaggggaaatagaatagaaatttcttacgaaaatttacaggtcacaaatattactagaaagggaagcatgttaccagtgagctgtaagttaccgaatatgggtacttcccagagttacctttcctcatctatgccagctggttgtgttttgccagtaggtggtaattttgagatcttgttacattactatcttctcaattatgccaaaaaatgccatcaatcagaagaaaccatggttagtatgataatagctaatgcacttttaggcattcccaaagtcctttataaatctaaaacaggaaagtacagctttccacatacatatataagagctgtccatgcactgcaaaccaatcaacccttggtaagcagtcagacaggtttggaatcagtaatgggtaaataccagctactaacttcagttcttcagtgtttgacaaaaatattaaccattgacatggtaatcactgttaagagacaccctcagaaagttcacaatcaagattcagaagatgaactataagcggccgcgtcgac

SEQ ID NO: 43 - DNA, 2898 bp, synthetic construct
  misc_feature 1..2898: note = Construct comprising CAG promoter and COSEQ1-BBS10 nucleotide sequence (referred to as CAG-COSEQ1-BBS10)
  source 1..2898: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatgctgtctagcatggctgccgctggatctgtgaaagctgctctgcaggttgccgaggtgctggaagccatcgtgtcttgttgtgtgggacctgagggcagacaggtgctgtgtacaaagcctacaggcgaagtgctgctgagcagaaatggcggcagactgctcgaagccctgcacctggaacaccctatcgccagaatgatcgtggactgcgtgtccagccacctgaagaaaacaggcgacggcgccaagaccttcatcatctttctgtgccatctgctgcggggcctgcacgccatcaccgatagagaaaaggaccctctgatgtgcgagaacatccagacacacggccggcactggaagaactgctcccggtggaagtttatcagccaggctctgctgacctttcagacccagatcctggacggcatcatggaccagtacctgagcagacacttcctgagcatcttcagcagcgccaaagaacggaccctgtgcagaagctctctggaactgctgctcgaggcctacttttgcggcagagtgggcagaaacaaccacaagttcatctcccagctgatgtgtgactacttcttcaagtgcatgacctgcaagagcggcatcggcgtgttcgagctggtggacgatcacttcgtggaactgaatgtgggcgtgaccggcctgcctgtgtccgatagcagaattattgccggcctggtgctgcagaaagacttcagcgtgtacagacccgccgacggcgacatgagaatggtcatcgtgaccgagacaatccagcctctgttcagcacaagcggcagcgagttcatcctgaacagcgaggcccagtttcagaccagccagttctggatcatggaaaagaccaaggccatcatgaagcacctccacagccagaacgtgaagctgctgatctccagcgtgaagcagcccgacctggtgtcttattatgccggcgtgaacggcatcagcgtggtggaatgtctgagcagcgaagaggtgtccctgatcagacggatcatcggactgagcccctttgtgcctcctcaagcctttagccagtgcgagatccctaacacagccctggtcaagttctgcaagcccctgatcctgcggagcaagagatatgtgcacctgggcctgatcagcacatgcgccttcattcctcactccatcgtgctgtgtggacctgtgcacggactgattgagcagcacgaagatgcactgcacggcgccctgaaaatgctgagacagctgttcaaggacctggacctgaactacatgacccagaccaacgaccagaacggcaccagcagcctgttcatctacaagaacagcggcgagagctatcaggccccagatccaggcaatggcagcatccagaggccttaccaggataccgtggccgagaacaaggacgccctggaaaaaacccagacctacctgaaggtgcacagcaacctggtcatccccgatgtggaactggaaacctacattccctacagcacccctacactgacccctaccgataccttccagaccgtggaaaccctgacctgtctgagcctggaacggaacagactgaccgactactacgagcccctgctgaaaaacaacagcaccgcctatagcacccggggcaacagaatcgagatcagctacgagaacctgcaagtgaccaacatcacccggaagggctccatgctgccagtgtcctgcaagctgcctaatatgggcaccagccagagctacctgtcctcttctatgcctgccggatgtgtgctgcctgtcggcggcaattttgagatcctgctgcactactacctgctgaactacgccaagaagtgccaccagagcgaagagacaatggtgtccatgattatcgccaacgctctgctgggcatccccaaggtgctgtacaagagcaagaccggcaagtacagcttccctcacacctacattagagccgtgcacgccctgcagaccaatcagccactggtttctagccagacaggcctggaaagcgtgatgggaaagtaccagctgctgaccagcgtgctgcagtgcctgaccaagatcctgaccatcgacatggtcatcaccgtgaagcggcaccctcagaaagtgcacaaccaggacagcgaggacgagctgtaggcgccgtcgac

SEQ ID NO: 44 - DNA, 2901 bp, synthetic construct
  misc_feature 1..2901: note = Construct comprising CAG promoter and COSEQ2-BBS10 nucleotide sequence (referred to as CAG-COSEQ2-BBS10)
  source 1..2901: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatgctgagctccatggctgccgccggatctgtgaaagccgccctgcaggtggcagaggtgctggaggcaatcgtgagctgttgcgtgggacctgagggccggcaggtgctgtgcacaaagccaaccggcgaggtgctgctgtctagaaatggcggccggctgctggaggccctgcacctggagcacccaattgcaagaatgatcgtggactgcgtgtctagccacctgaagaagacaggcgatggcgccaagaccttcatcatcttcctgtgccacctgctgaggggcctgcacgccatcacagaccgcgagaaggatcctctgatgtgcgagaacatccagacccacggcaggcactggaagaattgttcccgctggaagttcatctctcaggccctgctgacatttcagacccagatcctggacggcatcatggatcagtatctgagcaggcacttcctgtccatcttttcctctgccaaggagcggaccctgtgcagaagctccctggagctgctgctggaggcctacttctgtggccgggtgggcagaaacaatcacaagtttatcagccagctgatgtgcgactatttctttaagtgcatgacctgtaagtccggcatcggcgtgttcgagctggtggacgatcactttgtggagctgaacgtgggagtgacaggcctgcccgtgtccgactctcgcatcatcgccggcctggtgctgcagaaggatttctccgtgtaccggcctgccgacggcgatatgagaatggtcatcgtgaccgagacaatccagccactgttcagcacctccggctctgagttcatcctgaacagcgaggcccagttccagacatctcagttttggatcatggagaagaccaaggccatcatgaagcacctgcacagccagaacgtgaagctgctgatctctagcgtgaagcagccagacctggtgtcttactatgccggcgtgaatggcatcagcgtggtggagtgtctgtcctctgaggaggtgtccctgatccggagaatcattggcctgtctcccttcgtgccccctcaggcctttagccagtgcgagatccccaacacagccctggtgaagttctgtaagcctctgatcctgaggtccaagcggtacgtgcacctgggcctgatcagcacctgcgcctttatcccacactctatcgtgctgtgcggacctgtgcacggcctgattgagcagcacgaggatgcactgcacggcgccctgaagatgctgaggcagctgttcaaggacctggatctgaattacatgacccagacaaacgaccagaatggcacaagctccctgtttatctacaagaactctggcgagagctatcaggccccagatcccggcaatggcagcattcagcgcccctaccaggacacagtggcagagaacaaggatgccctggagaagacccagacatatctgaaggtgcactccaacctggtcatccctgacgtggagctggagacatacatcccttattctaccccaacactgacccccacagataccttccagacagtggagacactgacctgcctgtccctggagaggaaccgcctgaccgactactatgagcccctgctgaagaacaattccacagcctactctacccggggcaatagaatcgagatcagctatgagaacctgcaggtgacaaatatcaccagaaagggctctatgctgcctgtgagctgcaagctgccaaacatgggcaccagccagtcctacctgtctagctccatgcctgcaggatgcgtgctgcctgtgggcggcaacttcgagatcctgctgcactactatctgctgaattatgccaagaagtgccaccagagcgaggagacaatggtgtccatgatcatcgccaacgccctgctgggcatcccaaaggtgctgtacaagtctaagaccggcaagtacagctttccccacacctatatccgggccgtgcacgccctgcagacaaatcagcctctggtgtctagccagaccggcctggagtccgtgatgggcaagtaccagctgctgacatctgtgctgcagtgtctgacaaagatcctgaccatcgatatggtcatcaccgtgaagagacacccacagaaggtgcacaatcaggacagcgaggatgagctgtaagcggccgcgtcgac

SEQ ID NO: 45 - DNA, 204 bp, Human cytomegalovirus
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 46 - DNA, 232 bp, Homo sapiens
  source 1..232: mol_type = other DNA
ggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 47 - DNA, 528 bp, synthetic construct
  misc_feature 1..528: note = CAG promoter sequence
  source 1..528: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 48 - DNA, 1170 bp, Homo sapiens
  source 1..1170: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 49 - DNA, 278 bp, Gallus gallus
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg